(19) United States
(12) Patent Application Publication
(10) Pub. No.: US 2024/0418715 A1
(43) Pub. Date: Dec. 19, 2024

(54) URINARY BIOMARKERS FOR ACTIVE LUPUS NEPHRITIS AND METHODS OF USE THEREOF

(71) Applicant: UNIVERSITY HEALTH NETWORK, Toronto (CA)

(72) Inventors: Joan WITHER, Toronto (CA); Laura WHITTALL-GARCIA, Toronto (CA)

(21) Appl. No.: 18/677,065

(22) Filed: May 29, 2024

(30) Foreign Application Priority Data
May 29, 2023 (CA) 3200956

(51) Int. Cl. G01N 33/564; G01N 33/68

(52) U.S. Cl. CPC G01N 33/564; G01N 33/6863; G01N 33/6872; G01N 2800/104; G01N 2800/347

(57) ABSTRACT

Provided are urinary biomarker panels, kits and devices for detecting active Lupus Nephritis (ALN) comprising a plurality of biomarker detection agents, wherein each biomarker detection agent is specific for a corresponding target biomarker, the target biomarkers consisting of Adiponectin, MCP-1, sVCAM-1, and PF4, and none, one or both of vWF and IL-15, and optionally one or more control biomarkers. Also provided are methods and uses of the target biomarkers including methods of detecting acute lupus nephritis (ALN), monitoring ALN, predicting treatment outcome and treating ALN.

### CROSS-REFERENCE TO RELATED APPLICATIONS

This application claims priority from Canadian patent application number 3200956 filed May 29, 2023; this application being incorporated herein in its entirety by reference.

### FIELD

This disclosure pertains to biomarkers associated with active Lupus Nephritis (ALN) and methods and products for detecting ALN biomarker levels, predicting ALN treatment outcomes, detecting ALN, selecting ALN treatments and monitoring and/or treating ALN.

### BACKGROUND

Lupus nephritis (LN) occurs in up to 65% of patients with Systemic Lupus Erythematosus (SLE), and is most prevalent in younger patients, many of whom are of African, Asian, and Hispanic ancestry (1-3). LN is one of the most common causes of death as well as an important predictor of subsequent mortality in SLE (3-8). It is also associated with a significant morbidity, since up to 20% of patients will progress to end stage renal disease (3, 9), which has a particularly high socioeconomic impact (10, 11).

The gold standard for determining the presence and type of kidney involvement is the kidney biopsy (KB) (12). However, serial biopsies to assess renal activity following treatment are impractical due to their invasive nature and risk of complications. There is also a subset of patients with contraindications that preclude a KB at the time of LN flare. Consequently, the diagnosis of LN and the monitoring of response to treatment has been based on urinary findings of proteinuria, hematuria, pyuria, or casts, and alterations of renal function, such as increased serum creatinine.

The utility of proteinuria as a biomarker has drawbacks. LN-associated proteinuria frequently persists for years after renal injury, especially in patients with nephrotic range proteinuria, normalizing in less than 50% of patients within two years (13). In addition, proteinuria may reflect chronic histologic lesions rather than active inflammation within the kidney, as demonstrated by Malvar et al. who showed that 62% of LN patients who had complete histologic remission on a repeat KB following initiation of therapy were still ‘clinically active’, as defined by persistent proteinuria (14). Being able to correctly differentiate between residual activity and damage in LN is crucial when treating patients, highlighting the need for new biomarkers in the clinical setting.

Various urinary cytokines, chemokines, pro-inflammatory factors, growth factors and adhesion molecules, have been assessed as potential urinary biomarkers for LN (15-24). Unfortunately, none of them have been able to successfully transition into clinical practice, with the lack of clear cut-offs and algorithms that accurately detect active LN (ALN) being part of the challenge. Previously 42 urine biomarkers (of 129 tested by Luminex) were identified as discriminating between ALN and non-LN patients (NLN). Of these, Clusterin, Cystatin C, NGAL, PF4, VWF, sVCAM-1, GM-CSF, GRO, IL-15, IL-6, MCP-1, Adiponectin, PAI-1, MMP-7 and TIMP-1 were the 15 biomarkers with the most promising results, based on their ability to discriminate between ALN and non-active LN (remission LN, RLN) and/or their correlations with histologic features in the KB (16).

The accuracy of individual biomarkers is low. Given the invasive nature of the KB, the current gold standard for LN diagnosis, and the known drawbacks of proteinuria, the most commonly used parameter for LN surveillance, there is a need for biomarkers that more accurately identify active LN cases.

### SUMMARY

An aspect includes a urinary biomarker panel for detecting active Lupus Nephritis (ALN) comprising a plurality of biomarker detection agents, wherein each biomarker detection agent is specific for a corresponding target biomarker, the target biomarkers consisting of Adiponectin, MCP-1, sVCAM-1, and PF4, and none, one or both of vWF and IL-15, and optionally one or more control biomarkers.

In some embodiments, each biomarker detection agent is an antibody or binding fragment thereof.

In some embodiments, the biomarker panel further comprises a solid support.

In some embodiments, the solid support is a bead, a plate, or a chip, optionally wherein the panel is configured as a multiplex assay or an ELISA assay.

In some embodiments, bead comprises a unique code, optionally a colour code, and each unique code is associated with a different biomarker detection agent.

Another aspect includes a kit comprising the biomarker panel described herein, and one or more of:
i) a sample dilution buffer;
ii) a wash buffer;
iii) a filter;
iv) a positive control; and/or
v) instructions for performing a method described herein.

Another aspect includes a device comprising:
i) a sample inlet configured for receiving one or more test urine samples; and
ii) a urinary biomarker panel, optionally the biomarker panel of described herein and configured to contact the test samples comprising a plurality of biomarker detection agents, wherein each biomarker detection agent is specific for a corresponding target biomarker selected from the group consisting of Adiponectin, MCP-1, sVCAM-1, and PF4, and none, one or both of vWF and IL-15, and optionally one or more control biomarkers.

In some embodiments, the panel, kit or device is for detecting active Lupus Nephritis (ALN), optionally for detecting proliferative Lupus Nephritis (PLN).

Another aspect includes a method of assessing treatment response and/or predicting a treatment outcome in a subject that has active Lupus Nephritis (ALN) comprising:
i) acquiring one or more test urine samples from the subject, wherein the test samples are acquired within about 6 to about 15 months following onset of administration of an ALN treatment or LN flare,
ii) measuring levels of a plurality of target biomarkers in the test samples, comprising contacting the test samples with a urinary biomarker panel comprising a plurality of biomarker detection agents, optionally the urinary biomarker panel described herein, wherein each biomarker detection agent is specific for a corresponding target biomarker and the plurality of target biomarkers comprise Adiponectin, MCP-1, sVCAM-1, and PF4, and none, one or both of vWF and IL-15;
iii) comparing the measured levels of each of the test sample target biomarkers to a control, optionally a base line level or cut-off level; and
iv) assessing response or predicting the treatment outcome, wherein the subject is having or has an increased probability of having a complete response to the treatment if the measured levels for at least two, at least three, or each target biomarker are decreased compared to the control and/or less than two target biomarkers are decreased compared to the control optionally reach a cut-off level;
In some embodiments, the method comprises:
if at least two of the target biomarkers are Adiponectin, MCP-1, sVCAM-1 and/or PF4 and the measured levels of the two biomarkers do not reach the cut-off levels, and the cut off level for sVCAM-1 is a threshold level, the subject is identified as having or likely to have a complete response to the treatment; and
if at least two of the target biomarkers are Adiponectin, MCP-1, sVCAM-1 and/or PF4 and the measured levels of the said at least two biomarkers reach the cut-off levels, wherein if the target biomarkers that reach the cut-off are two biomarkers and the two biomarkers includes sVCAM-1 and not MCP-1, the cut-off level of sVCAM-1 is an elevated cut off level, the subject is identified as less likely to have a complete response to the treatment.

In some embodiments, the control is a cut-off level determined from levels from one or more subjects having ALN and known treatment outcomes and/or healthy control subjects and/or wherein the treatment outcome is at about 24 months.

Another aspect includes a method of detecting active Lupus Nephritis (ALN) in a subject comprising:
i) acquiring one or more test urine samples from the subject;
ii) measuring levels of a plurality of target biomarkers in the test samples, comprising contacting the test samples with a urinary biomarker panel comprising a plurality of biomarker detection agents, optionally the urinary biomarker panel described herein, wherein each biomarker detection agent is specific for a corresponding target biomarker and the plurality of target biomarkers comprise Adiponectin, MCP-1, sVCAM-1, and PF4, and none, one or both of vWF and IL-15;
iii) comparing the measured levels of each of the test sample target biomarkers to a cut-off level for each target biomarker; and
iv) detecting whether the subject has ALN or does not have ALN, wherein if at least two of the target biomarkers are Adiponectin, MCP-1, sVCAM-1 and/or PF4 and the measured levels of the two biomarkers do not reach the cut-off levels, and the cut off level for sVCAM-1 is a threshold level, the subject is identified as likely not having ALN; and
if at least two of the target biomarkers are Adiponectin, MCP-1, sVCAM-1 and/or PF4 and the measured levels of the said at least two biomarkers reach the cut-off levels, wherein if the target biomarkers that reach the cut-off are two biomarkers and the two biomarkers includes sVCAM-1 and not MCP-1, the cut-off level of sVCAM-1 is an elevated cut off level the subject is identified as likely having ALN.

In some embodiments, the cut-off level for each target biomarker is:
a) about 18 ng/ml for Adiponectin or about or at least 3 fold over a SLE NLN control population level;
b) about 1.3 ng/mL for MCP-1 or about or at least 5 fold over a SLE NLN control population level;
c) about 46 ng/mL for sVCAM-1 or about or at least 2 fold over a SLE NLN control population level; and/or
d) about 0.13 ng/ml for PF4 or about or at least 2.3 fold over a SLE NLN control population level.

In some embodiments, at least three of the target biomarkers are Adiponectin, MCP-1, sVCAM-1 and/or PF4 and measured levels for each of the said three target biomarkers reach the cut-off levels and indicate the subject likely has ALN or is unlikely to have a complete response to treatment.

In some embodiments, wherein the at least two of the target biomarkers are:
e) Adiponectin and MCP-1;
f) Adiponectin and PF4;
g) MCP-1 and PF4; or
h) MCP-1 and sVCAM-1.

In some embodiments, the at least two of the target biomarkers are:
j) sVCAM-1 and Adiponectin; or
k) sVCAM-1 and PF4; and
the cut off level for sVCAM-1 is the elevated cut-off level.

In some embodiments, the cut-off level for sVCAM-1 is about 104 ng/ml or about or at least 4.4 fold over a SLE NLN control population level.

In some embodiments, wherein the test samples is taken from a subject that has had a Lupus Nephritis (LN) flare within 12 or within 15 months from when the test samples were acquired and/or when the subject is predicted to have ALN, the method further comprises administering a treatment for ALN to the subject.

A further aspect is a method of treating active Lupus Nephritis (ALN) comprising:
in a subject receiving an ALN treatment, adjusting the ALN treatment in a subject having increased or comparable measured levels of at least two of a plurality of target biomarkers relative to a baseline level and/or control in one or more test urine samples, optionally as assessed or predicted as described herein as less likely to have a complete response, or continuing administration of the ALN treatment in a subject that has decreased measured levels of at least two of a plurality of target biomarkers relative to a baseline level and/or control in one or more test urine samples optionally as assessed or predicted as described herein as likely to have a complete response, wherein the target biomarkers comprise Adiponectin, MCP-1, sVCAM-1, and PF4, and none, one or both of vWF and IL-15 and wherein the measured levels are from samples taken within 6 to 15 months from onset of the treatment, optionally wherein adjusting the ALN treatment comprises discontinuing administration of the ALN treatment and initiating administration of an alternate ALN treatment, adjusting a dose of a component of the ALN treatment or adding a therapeutic; or
in a subject not receiving treatment for ALN, administering an ALN treatment, optionally comprising azathioprine, cyclophosphamide or mycophenolate, to a subject having measured levels of at least two target biomarkers of a plurality of biomarkers reach a cut-off level for each of the said at least two target biomarkers, wherein the target biomarkers comprise Adiponectin, MCP-1, sVCAM-1 and/or PF4, and none, one or both of vWF and IL-15, optionally according to the method described herein.

In some embodiments, the test samples are taken at about 6, or about 9 or about 12 or about 15 months from onset of the treatment.

A further aspect includes a method of monitoring and/or treating a subject with or suspected of having active Lupus Nephritis (ALN) comprising:
i) acquiring one or more test urine samples from the subject;
ii) measuring levels of a plurality of target biomarkers in the test samples, comprising contacting the test samples with a urinary biomarker panel comprising a plurality of biomarker detection agents, optionally the urinary biomarker panel described herein, wherein each biomarker detection agent is specific for a corresponding target biomarker and the plurality of target biomarkers comprise Adiponectin, MCP-1, sVCAM-1, and PF4, and none, one or both of vWF and IL-15, and optionally one or more control biomarkers;
iii) comparing the measured levels of each of the test sample target biomarkers to levels of the target biomarkers in one or more control samples, optionally wherein the control samples are baseline samples acquired from the subject or are acquired from one or more healthy control subjects; and
iv) a) administering an ALN treatment based on the measured levels of the target biomarkers in the test samples, wherein at least two of the target biomarkers are Adiponectin, MCP-1, sVCAM-1 and/or PF4 and the measured levels for the said target biomarkers reach a cut-off level for each target biomarker, optionally wherein the treatment comprises administering azathioprine, cyclophosphamide or mycophenolate; or
b) adjusting the ALN treatment, optionally discontinuing administration of the ALN treatment and administering an alternate ALN treatment based on the measured levels of the target biomarkers in the test samples, wherein at least two of the target biomarkers are Adiponectin, MCP-1, sVCAM-1 and/or PF4 and the measured levels for the said target biomarkers reach a cut-off level for each target biomarker; and/or
c) directing a follow up testing repeating steps i) to iii) to monitor the subject.

In some embodiments, the adjusted treatment or the alternate treatment azathioprine, cyclophosphamide or mycophenolate and/or comprises tacrolimus, voclosporin and/or belimumab.

In some embodiments, the cut-off level for each target biomarker is:
a) about 18 ng/ml for Adiponectin or about or at least 3 fold over a SLE NLN control population level;
b) about 1.3 ng/ml for MCP-1 or about or at least 5 fold over a SLE NLN control population level;
c) about 46 ng/ml for sVCAM-1 or about or at least 2 fold over a SLE NLN control population level; and/or
d) about 0.13 ng/ml for PF4 or about or at least 2.3 fold over a SLE NLN control population level.
wherein at least three of the target biomarkers are Adiponectin, MCP-1, sVCAM-1 and/or PF4 and measured levels for each of the said three target biomarkers reach the cut-off levels and indicate adjusting the ALN treatment and/or administering the ALN treatment;
or wherein the at least two target biomarkers are:
e) Adiponectin and MCP-1;
f) Adiponectin and PF4;
g) MCP-1 and PF4; or
h) MCP-1 and sVCAM-1.

In some embodiments, the target biomarkers are at least three target biomarkers, selected from for example Adiponectin, MCP-1, sVCAM-1 and/or PF4 or the target biomarkers are:
j) sVCAM-1 and Adiponectin; or
k) sVCAM-1 and PF4;
optionally wherein the cut off level for sVCAM-1 is the elevated cut-off level, optionally wherein the cut-off level for sVCAM-1 is about 104 ng/mL or about or at least 4.4 fold over a SLE NLN control population level.

In some embodiments, wherein the ALN is proliferative Lupus nephritis (PLN).

In some embodiments, the cut-off level of each target biomarker is selected to provide a desired sensitivity, specificity, positive predictive value (PPV), negative predictive value (NPV), positive likelihood ratio (+LR) and/or negative likelihood ratio (−LR).

In some embodiments, the selected specificity, and/or NPV for each target biomarker is above about 90% or about 95%, and/or the PPV is above about 95% or about 100%.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the present disclosure are described with reference to the drawings:

FIG. 1 shows thumbnail plots illustrating the difference in the amount of urinary biomarkers over time between complete responders (CR; n=12, blue) and partial or non responders (PR or NR; n=9, red). Units for all graphs are in pg/mmol, except PF4 expressed in ng/mmol.

FIG. 2 shows a comparison of biomarker levels between active LN (ALN; n=24, circles), remission LN (RLN; n=79, squares) and non LN (NLN; n=144, triangles). For all graphs each symbol represents the determination from a single individual, with the median value for each group indicated by a horizontal line. The Kruskal-Wallis test was used to assess the differences in biomarker levels between ALN, RLN and NLN patients.

FIG. 3 shows Receiver Operating Characteristic curves for Adiponectin, MCP-1, sVCAM-1 and PF 4 urinary biomarkers.

FIG. 4 shows a 2 step approach for the detection of ALN patients as herein disclosed.

### DETAILED DESCRIPTION OF THE DISCLOSURE

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

The term “adiponectin” as used herein includes naturally occurring variants of the polypeptide having uniprot accession number Q15848.

The term “MCP-1” as used herein means “monocyte chemotactic protein 1” and includes naturally occurring variants of the polypeptide having uniprot accession number Q6UZ82.

The term “sVCAM-1” as used herein means “soluble vascular (cell) adhesion molecule 1” and includes naturally occurring variants of the polypeptide having accession number uniprot P19320.

The term “PF4” as used herein means “platelet factor 4” which is synonymous with “CXCL4” (chemokine C-X-C motif ligand 4) and includes naturally occurring variants of the polypeptide having uniprot accession number P02776.

The term “vWF” as used herein means “von Willebrand factor” and includes naturally occurring variants of the polypeptide having uniprot accession number P04275

The term “IL-15” as used herein means “interleukin 15” and includes naturally occurring variants of the polypeptide having uniprot accession number P401933.

The term “PAI-1” as used herein means “plasminogen activator inhibitor-1”.

The term “GM-CSF” as used herein means “granulocyte macrophage colony-stimulating factor”.

The term “GRO” as used herein means “growth related oncogene”.

The term “MMP-7” as used herein means “matrix metalloproteinase 7”.

The term “IL-6” as used herein means “interleukin 6”.

The term “TIMP-1” as used herein means “TIMP metallopeptidase inhibitor 1”.

The term “active Lupus Nephritis (ALN)” as used herein means Systemic Lupus Erythematosus (SLE) patients who have active lupus nephritis, including, for example, patients diagnosed with Lupus Nephritis (“LN”) for example, based on kidney biopsy (“biopsy-proven”) and/or based on urinary findings of proteinuria, hematuria, pyuria, or casts, and alterations of renal function, such as increased serum creatinine. ALN can be diagnosed by new onset of urinary proteinuria >0.5 g per day with or without the presence in urine of cellular casts including red cell or heme-granular casts, >5 RBC/high power field (HPF) or >5 WBC/HPF, in the absence of infection or kidney stones. A renal biopsy demonstrating immune complex-mediated glomerulonephritis compatible with lupus nephritis is also diagnostic of ALN. Proliferative LN (PLN), which is also referred to as class III and class IV (both either with or without class V), is a subset of ALN that is highly inflammatory with immune complex deposition in the subendothelial space. PLN can be distinguished from pure class V or membranous LN which is less inflammatory than PLN and is characterized by immune complex deposition in the subepithelial space.

The term “RLN” as used herein means SLE patients who have a history of LN but are in remission.

The term “non-lupus nephritis (NLN)” or “non-LN” as used herein means SLE patients that do not have LN and/or that do not have a history of LN.

The term “reach” with respect to cut-off levels as used herein means meeting or being of comparable level to or surpassing or exceeding the cut-off level. For example, in the context of increased measured levels of target biomarkers, reaching a cut-off level of about 18 ng/ml for Adiponectin means the measured level is or corresponds to about 18 ng/ml or is greater than 18 ng/ml. By contrast, a measured level of target biomarker that does not reach a cut-off level of about 18 ng/ml for Adiponectin means the measured level is less than about 18 ng/ml.

The term “subject” as used herein means all members of the animal kingdom including mammals, preferably humans.

The term “antibody” as used herein is intended to include monoclonal antibodies, polyclonal antibodies, chimeric antibodies, humanized antibodies as well as human antibodies, identified for example using phage display, and antibody binding fragments thereof. The antibody may be from recombinant sources and/or produced in transgenic animals. The term “antibody binding fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

The term “baseline level” as used herein means an initial or reference level of measured target biomarker in urine samples taken from the same subject test urine samples are taken from. For example, a baseline level of target biomarkers may be measured soon before, concurrently with, or shortly after onset of administration of a treatment.

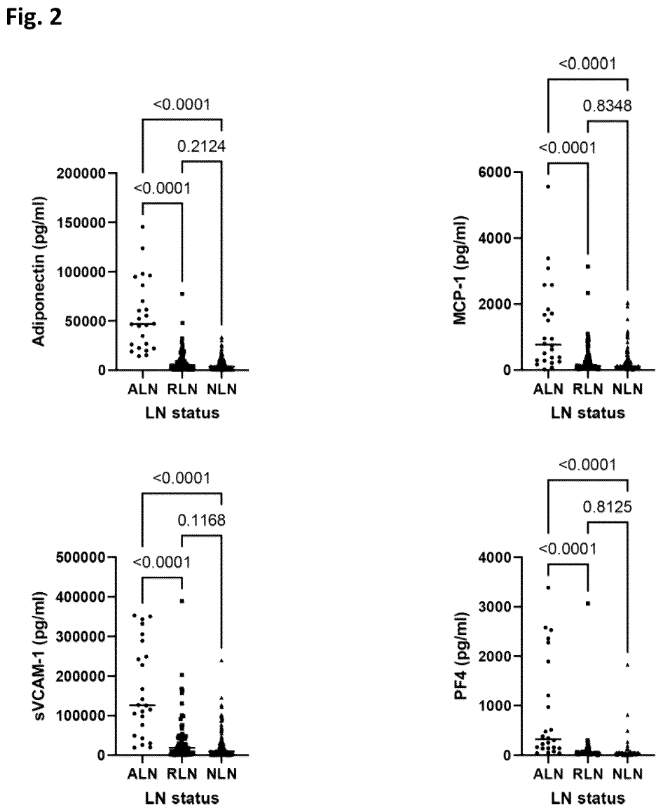

The term “control” as used herein means a sample from a suitable control, for example obtained from a subject without a condition being assessed (e.g. healthy control, NLN, or RLN) and/or a subject with a known outcome, or a plurality of samples from subjects without the condition being assessed and/or with known outcomes from which a reference value e.g. a cut-off value is determined. The control can also be a reference value such as a cut-off value, or set of reference values such as a threshold cut-off value and an elevated cut-off value. The reference value(s) can be a selected level such as an average level or median level exhibited for example in a clinical population that does not exhibit the condition or disease to be detected, for example, RLN or patients in remission for LN, or NLN or non-LN patients or a reference value that provides a selected sensitivity, specificity NPV and/or PPV. In methods for predicting disease outcome, the reference level can be a prior level of the subject, for example a baseline level, or based on known outcome samples, for example in one or more kidney biopsy (KB)-proven ALN patients. The reference value for a biomarker can, for example, be determined by reference to the corresponding control values depicted in FIG. 2 and/or determined by similar methods as described herein in other control populations.

The term “complete response” as used herein with respect to ALN means resolving of inflammation detectable by a decrease or absence of proteinuria related to inflammation. For example, complete remission includes a subject with less than <0.5 g/24 hrs urinary protein and creatinine levels that are normal or within 15 percent of baseline. A complete response can interchangeably be referred to in complete remission.

The term “partial response” as used herein means a lessening of proteinuria to less than 50% of baseline and to non-nephrotic levels (<3 gms/24 hrs) with a serum creatinine within 25% of previous baseline. Treatment failure can be the inability to achieve either a complete or partial response.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 5-40%, 5-20%, 5%-15%, preferably 5-10%, most preferably about 5% or about 10% of the number to which reference is being made.

As used in this specification and the appended claims, the singular forms “a”, “an” and “the” include plural references unless the content clearly dictates otherwise. Thus, for example, “a biomarker” includes “two biomarkers”. It should also be noted that the term “or” is generally employed in its sense including “and/or” unless the content clearly dictates otherwise.

The term “one or more” as used herein means 1, 2, 3, etc., up to the number of elements of the set, Table or Figure. For example, one or more control biomarkers includes any one, any two, any three, etc., control biomarkers.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### I. Urinary Biomarker Panels

The present disclosure identifies biomarkers that can with high accuracy identify patients with active Lupus Nephritis (ALN), and patients with ALN who are responding to treatment. The ALN biomarkers include Adiponectin, MCP-1, sVCAM-1, and PF4. Provided herein are methods and products that can be used for detecting levels of said ALN biomarker levels, predicting ALN treatment outcomes, detecting ALN, and monitoring and/or treating ALN. Methods and products as described herein can be carried out or made inexpensively relative to known methods and products in the art for detecting ALN biomarker levels, predicting ALN treatment outcomes, detecting ALN, selecting ALN treatments and monitoring and/or treating ALN.

In an aspect, a urinary biomarker panel for detecting active Lupus Nephritis (ALN) as herein disclosed comprises a plurality of biomarker detection agents. Each biomarker detection agent of the panel is specific for a corresponding target biomarker, and the target biomarkers consist of Adiponectin, MCP-1, sVCAM-1, and PF4, and none, one or both of vWF and IL-15. Optionally, the panel may also include one or more controls, such as control biomarkers or control biomarker detection agents for detecting urinary components such as urinary creatinine or urinary proteins. The control biomarker can also be a positive control such as an amount or amounts of one or more of the target biomarkers. In some embodiments, the biomarker panel lacks a normalization control. In other embodiments, the biomarker panel includes a normalization control.

In some embodiments, the biomarker detection agent may be an antibody which is specific for a corresponding target biomarker. Other detection binding agents may also be used. The detection binding agent may be labelled, or uniquely coded, for example with a fluorescent molecule, pigment, or other signal inducing molecule or may be indirectly detectable using for example a secondary antibody. Other labels such as metal tag can be used. Metal tagged antibodies, for example, can be detected by mass cytometry.

In further embodiments, the biomarker panels as herein disclosed further comprise a solid support, which may be any solid means known in the art on which a biomarker detection agent, for example an antibody or antibody binding fragment thereof, can be coated or affixed. In some embodiments, the solid support is one or more beads, which may each be uniquely coded, for example, colour-coded with a fluorescent molecule or pigment or other signal inducing molecule for identification of the biomarker detecting agent that the solid support is affixed to and the target biomarker that specifically associates with the biomarker detecting agent. In other embodiments, the solid support is a plate (e.g. wells of a plate) on which the biomarker detection agents are attached or a solid support that is configured to bind to or be coated with the biomarker detection agents, or a chip on which the biomarker detection agents are attached, for example an electronic device configured for detecting and/or measuring light or electrochemiluminescence signals.

In some embodiments, the biomarker panels are configured as a multiplex assay or an ELISA assay. The biomarker panel can be used in the kits, devices, methods or uses described herein.

### II. Kits

In an aspect, a kit as herein disclosed comprises a biomarker panel as herein disclosed, and at least one or more of a sample dilution buffer, a wash buffer, a filter, a positive control or nonspecific/background control and/or instructions for performing any method as described herein.

For example, the positive control can be the target biomarker that can be serially diluted. They may also be provided serially diluted. Such controls can be used to generate a standard curve.

The kit may also comprise a sterile urine collection tube in some embodiments.

The kits can be for use of a method described herein. The kit can be for processing a sample or for processing a plurality of samples optionally from a plurality of subjects. For example the dilution buffer, wash buffers, filters may be for one sample or a plurality such as 10 samples, 20 samples, 50 samples or more.

The kits can be used in the devices, methods or uses described herein.

### III. Devices

In an aspect, a device comprising the urinary biomarker panel is also provided. The device in some embodiments is for detecting active Lupus Nephritis (ALN). The device can be for use in a method as herein described. In some embodiments, the device comprises a sample inlet configured for receiving one or more test urine samples, and a urinary biomarker panel, which may be any biomarker panel as herein described. The biomarker panel is configured to receive or contact the test samples and comprises a plurality of biomarker detection agents. Each biomarker detection agent is specific for a corresponding target biomarker selected from the group consisting of Adiponectin, MCP-1, sVCAM-1, and PF4, and none, one or both of vWF and IL-15. Optionally, the panel may also include one or more control biomarkers e.g. positive controls, or non-specific controls.

In some embodiments, the devices as herein disclosed are for use for detecting proliferative Lupus Nephritis (PLN).

In some embodiments, a device for detecting active Lupus Nephritis (ALN) as herein described measures levels of target biomarkers in the test samples, compares the measured levels of each of the test sample target biomarkers to a cut-off level for each target biomarker, and predicts a probability that the subject has ALN, wherein the subject has an increased probability of ALN if at least two of the target biomarkers are Adiponectin, MCP-1, sVCAM-1 and/or PF4 and the measured levels of the said at least two biomarkers reach the cut-off levels relative to a subject having levels of the target biomarkers that are less than the cut-off levels as further described in the methods and uses. In some embodiments, the cut-off levels can be any cut-off levels to provide a desired PPV, NPV, sensitivity or specificity, for example as herein described.

The devices can be used in the methods and uses described herein.

### IV. Methods and Uses

In the methods and uses as herein disclosed, where a method, treatment or use is described in relation to ALN, it is to be understood that PLN is included in the term “ALN” and therefore the described method, treatment or use is also disclosed in relation to PLN.

In an aspect, a method of predicting treatment outcome is provided.

In an embodiment, the method assessing treatment response and/or predicting a treatment outcome in a test subject that has active Lupus Nephritis (ALN) as herein disclosed comprises the following steps: i) acquiring one or more test urine samples from the subject within about 6 to about 15 months, preferably at about 12 months, following onset of administration of a treatment for ALN; ii) measuring levels of a plurality of target biomarkers in the test samples, wherein the plurality of target biomarkers comprise at least Adiponectin, MCP-1, sVCAM-1, and PF4, and none, one or both of vWF and IL-15; iii) comparing the measured levels of each of the test sample target biomarkers to a control; and iv) assessing response and/or predicting the ALN treatment outcome based on the measured target biomarker levels. In an embodiment, the subject is having, or has an increased probability of having, a complete response to the treatment if the measured levels for at least two, at least three or each target biomarker are decreased compared to control and/or less than two target biomarkers reach a cut-off level. The subject can be assessed as responding if levels of the target biomarkers have decreased sufficiently or normalized (e.g. are below the cut-off levels). For example, the subject is having or has an increased chance of complete response if the subject's levels are normalized (e.g. similar to control levels such as levels seen in SLE NLN patients or similar to pre-flare levels (e.g. baseline)). If two or more than two of the target biomarkers reach the cut-off level, the subject is likely to have a partial response or no response depending on the level (e.g. if the subject's levels are closer to normal levels, the subject is more likely to have a partial response than a subject who does not experience a change from prior to treatment levels). If the levels are unchanged from the start of treatment (e.g. baseline), the subject is likely to have no response.

In an embodiment, the method comprises contacting the test sample(s) with a urinary biomarker panel which comprises a plurality of biomarker detection agents, which may be any urinary biomarker panel as herein disclosed, wherein each biomarker detection agent is specific for a corresponding target biomarker.

In an embodiment, the control is a baseline level, optionally the subject's baseline level or a normal level for a control population such as SLE NLN, SLE RLN or healthy controls or a cut-off determined from one or more control populations that provides for example a selected level of accuracy e.g. sensitivity or specificity or predictive power e.g. PPV or NPV.

The ALN treatment the subject is receiving or in methods where a treatment is adjusted or started, may be any treatment that is approved or used for treating for ALN, The treatment may comprise azathioprine, cyclophosphamide or mycophenolate. In some embodiments, the main therapeutic agent is azathioprine, cyclophosphamide or mycophenolate. The treatment may also comprise one or more other therapeutics such as belumimab, voclosporin, and/or tacrolimus.

Treatment response is typically assessed in ALN at about 24 months. A subject may have one or more of the ALN criteria (e.g. unresolved proteinuria) when the one or more urinary samples are obtained. A subject may be responding to treatment reflected in the combination of levels of the target biomarkers which are an early indication of whether the subject will be experience, a complete response at 24 months. If levels have normalized (e.g. returned to normal or fallen below the cut-off levels) for two or more of the target biomarkers as described herein and particularly if a subject has levels that have normalized for all four of the target biomarkers, the subject is responding to the treatment and has an increased likelihood compared to for example a subject where less than two of the target biomarkers have normalized, to having a complete response.

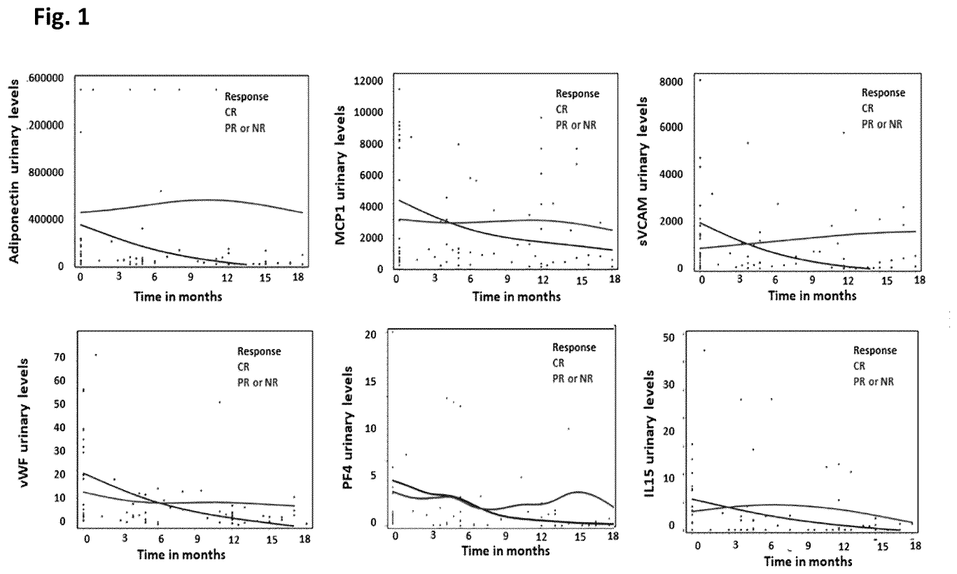

The decrease can be a compared to baseline levels. As shown herein, the percentage decrease in Adiponectin, MCP-1, sVCAM-1, PF4, IL-15 and vWF at 12±3 months predicted response to therapy at 24 months (see for example FIG. 1 and Table 2.

The one or more urinary samples can also be taken within 6 to 15 months from a LN flare. For example as demonstrated herein, subjects where urinary samples were assessed at about 12 months from a LN flare that met the cut off of criteria described herein, for example which had less than two target biomarkers that reached the cut-off level, had a high probability, for example greater than 90% chance, of complete response (e.g. 11 of 12 subjects that had less than 2 of the target biomarkers reach the cut-off level had a complete response at 24 months whereas only 4 of 10 subjects with greater than two target biomarkers reaching the target levels had a complete response).

In an embodiment, if two or more of the target biomarkers including Adiponectin, MCP-1, sVCAM-1 and/or PF4 do not reach the cut-off levels (i.e. only one or none of the target biomarkers reach the cut-off levels) and the cut off level for sVCAM-1 is a threshold level, the subject is identified as likely to have a complete response to the treatment; and/or if at least two of the target biomarkers are Adiponectin, MCP-1, sVCAM-1 and/or PF4 and the measured levels of the said at least two biomarkers reach the cut-off levels, wherein if the target biomarkers that reach the cut-off are two biomarkers and the two biomarkers includes sVCAM-1 and not MCP-1, the cut-off level of sVCAM-1 is an elevated cut off level, the subject is identified as less likely to have a complete response to the treatment.

In some embodiments, methods of assessing treatment response or predicting a treatment outcome in a test subject that has active Lupus Nephritis (ALN) as herein disclosed comprises: iv) predicting the ALN treatment outcome based on the measured target biomarker levels, wherein the subject has a decreased probability of having a complete response to the treatment if the measured levels for two or more, optionally 3 or each of the target biomarkers are increased compared to the control.

In some embodiments of any method herein described, the measured biomarker levels are normalized relative to a negative control, wherein measuring levels of target biomarkers in test sample(s) further comprises normalizing the measured levels of target biomarkers to a level of one or more control biomarkers. In other embodiments, the measured biomarker levels are non-normalized, wherein measuring levels of target biomarkers in test samples do not comprise normalizing the measured levels of target biomarkers to a level of one or more control biomarkers. For example, urinary creatinine may be used to normalize however any other urinary biomarker known for example to be relatively unchanged in ALN disease state may be used.

In some embodiments, the control may be based on levels of target biomarkers measured in samples acquired from kidney biopsy (KB)-proven ALN patients or KB-proven proliferative LN patients with known outcomes or cut-off levels determined from KB-proven ALN patients and/or KB-proven proliferative LN patients. The control can also be based on values of healthy control subjects and/or subjects with SLE NLN. The control can also be more than one control. For example, the methods, kits, devices and panels may comprise positive controls and negative controls. These may be provided as serial dilutions depending on the assay format.

In some embodiments, the control is a level determined from one or more subjects distinct from the test subject that have ALN and for whom an outcome of an administered treatment is known. A treatment outcome may include, for example, a complete response to the treatment, a partial response to the treatment, and/or no response to the treatment, as determined at a time following treatment initiation. In some embodiments, a treatment response is assessed at 24 months following treatment onset and/or LN flare.

In further embodiments, a method of assessing treatment response or predicting a treatment outcome may be used to assess or predict whether the subject has proliferative Lupus Nephritis (PLN).

As described herein, accuracy of ALN detection can be increased by excluding subjects that have less than two elevated biomarkers, e.g. only one or none of Adiponectin, MCP-1, sVCAM-1, or PF4, using a threshold cut-off level for sVCAM-1. As shown herein, the threshold or lower cut-off for sVCAM-1 provides a high NPV and low-LR suggesting that subjects that have a sVCAM-1 level that is elevated but below the threshold cut-off level, have a low probability of ALN. If two of said biomarkers are elevated, including the following combinations Adiponectin-MCP-1, Adiponectin-PF4, MCP-1-PF4 and sVCAM-1-MCP-1, given the PPV and +LR, the diagnosis of ALN is very probable. If the combination of 2 elevated biomarker includes sVCAM-1-Adiponectin or sVCAM-1-PF4, then the elevated cut-off level of sVCAM-1 can be used. If three or four of said biomarkers are elevated, the subject is very likely to have ALN. In such embodiments, the threshold or elevated cut-off level of sVCAM-1 can be used.

The methods described herein can reduce the number of invasive procedures such as renal biopsies. The confirmative method for diagnosing LN and distinguishing proliferative LN from membranous LN is renal biopsy. Renal biopsies are currently recommended in patients with SLE who have for example increasing creatinine levels of an unknown cause, proteinuria at protein levels of ≥1 g per day (either in a 24-h urine specimen or on a spot protein/creatinine ratio [PCR]), or a combination of the following: proteinuria at protein levels of ≥0.5 g per day plus ≥5 red blood cells (RBCs) per high power field (HPF) or proteinuria at protein levels of ≥0.5 g per day plus cellular casts. Renal biopsies may not always be available or advisable in all patients. For example, patient with hypertension, low platelet counts, or anticoagulation. Further the invasiveness of renal biopsies is a strong deterrent. Untreated or poorly treated ALN, and particularly PLN, can lead to kidney damage or further kidney damage including end stage renal disease.

The present methods and products for example allow discrimination of subjects with active disease.

In another aspect, a method of detecting active Lupus Nephritis (ALN) in a subject as herein disclosed comprises the following steps: i) acquiring one or more test urine samples from the subject; ii) measuring levels of a plurality of target biomarkers in the test samples, comprising contacting the test samples with a urinary biomarker panel comprising a plurality of biomarker detection agents, optionally a urinary biomarker panel as herein disclosed, wherein each biomarker detection agent is specific for a corresponding target biomarker and the plurality of target biomarkers comprise Adiponectin, MCP-1, sVCAM-1, and PF4, and none, one or both of vWF and IL-15, and optionally one or more control biomarkers; iii) comparing the measured levels of each of the test sample target biomarkers to a cut-off level for each target biomarker; and iv) detecting whether the subject has ALN or does not have ALN, for example by predicting a probability that the subject has ALN.

In an embodiment, if at least two of the target biomarkers are Adiponectin, MCP-1, sVCAM-1 and/or PF4 and the measured levels of the two biomarkers do not reach the cut-off levels, the subject has a decreased likelihood of ALN.

In an embodiment, the subject has an increased probability of ALN if at least two of the target biomarkers are Adiponectin, MCP-1, sVCAM-1 and/or PF4 and the measured levels of the two biomarkers reach the cut-off levels relative to a subject having levels of the target biomarkers that are less than the cut-off levels.

In an embodiment, if the target biomarkers that reach the cut-off are two biomarkers and the two biomarkers includes sVCAM-1 and not MCP-1, the cut-off level of sVCAM-1 is an elevated cut off level the subject is identified as likely having ALN. If two biomarkers reach the cut off level, it can be considered that the Rule out criteria have been met e.g. the subject did not have less than 2 biomarkers that were elevated.

In some embodiments of any method herein disclosed wherein measured target biomarker levels are compared to or considered in relation to a cut-off level, the cut-off level for each target biomarker can be a predetermined level for the biomarker known to have useful predictive, evaluative and/or diagnostic utility as determined from urine samples from patients having known treatment outcomes, optionally biopsy-proven ALN patients and/or healthy subjects that do not have ALN.

In some embodiments, the test samples are diluted. In other embodiments, the test samples are undiluted. For example, the test sample may be diluted for measuring one target biomarker and not for another depending on the dynamic range of the assay platform. Multiple dilutions may also be performed to increase the test accuracy.

The cut-off level can be selected to provide a particular sensitivity or specificity, NPV, PPV, LR− and/or LR+.

In some embodiments, a cut-off level for Adiponectin is about 18 ng/ml, a cut-off level for MCP-1 is about 1.3 ng/ml, a cut-off level for sVCAM-1 is about 46 ng/ml and/or a cutoff level for PF4 is about 0.13 ng/ml.

In other embodiments, in methods as herein disclosed, the assessment and/or prediction is based on elevated levels of at least three of the target biomarkers, e.g. selected from Adiponectin, MCP-1, sVCAM-1 and/or PF4. In such embodiments measured levels for each of the said three target biomarkers reach cut-off levels for each target biomarker indicate the subject has an increased probability of having ALN.

In some embodiments, in methods as herein disclosed, at least two of the target biomarkers are the combinations Adiponectin and MCP-1, Adiponectin and PF4, MCP-1 and PF4, or MCP-1 and sVCAM-1.

In other embodiments, in methods as herein disclosed, at least three of the target biomarkers are Adiponectin, MCP-1, sVCAM-1 and/or PF4 and at least two of the target biomarkers are the combinations sVCAM-1 and Adiponectin or sVCAM-1 and PF4.

In yet further other embodiments, in methods as herein disclosed, the cut-off level for sVCAM-1 is about 104 ng/ml (e.g. the elevated cut-off) and at least two of the target biomarkers are the combinations sVCAM-1 and Adiponectin or sVCAM-1 and PF4.

In some embodiments, the target biomarker level indicative of lack of response to treatment or indicative of ALN is a fold increase over normal population levels for example of SLE NLN patients.

In some embodiments, the cut-off level indicative of treatment response or detecting is an increase of at least or about 3-fold compared to a control population level (e.g. SLE NLN) for adiponectin.

In some embodiments, the cut-off level indicative of treatment response or detecting is an increase of at least or about 5-fold or at least or about 5.5-fold compared to a control population level (e.g. SLE NLN) for MCP-1.

In some embodiments, the cut-off level indicative of treatment response or detecting is an increase of at least or about 2-fold compared to a control population level (e.g. SLE NLN) for sVCAM-1 threshold level and at least or about 4 or 4.4-fold for sVCAM-1 elevated level.

In some embodiments, the cut-off level indicative of treatment response or detecting is an increase of at least or about 2 or at least or about 2.3-fold compared to a control population level (e.g. SLE NLN) for PF-4.

A subject that reaches the fold increase in biomarker level compared to the control population is less likely to be responding to treatment or have a complete response and/or has ALN.

In some embodiments, in methods as herein disclosed, the test sample is collected at about 6 to about 15 months after a subject has had a Lupus Nephritis (LN) flare. In some embodiments, the method further comprises administering or adjusting a treatment for ALN to the subject, for example administering a treatment if the subject is not yet receiving treatment or adjusting treatment if the subject is predicted to not have a complete response to the treatment they are, at the time of obtaining the sample, receiving.

The methods may also be used for predicting LN fares. In some embodiments, the subject is monitored, if levels of 2 or more target biomarkers are increased relative to the subjects earlier or baseline levels, the subject may be at risk for LN flare. In such cases, the subject may be administered an immunosuppressive agent such as a steroid, to try to correct or decrease the elevated target biomarkers.

In another aspect, methods of treating active Lupus Nephritis (ALN) as herein disclosed comprise administering an ALN treatment for example comprising azathioprine, cyclophosphamide or mycophenolate to a subject. The treatment or adjusted treatment may comprise one or more additional therapeutic agents such as tacrolimus, voclosporin or belimumab. In some subjects, the treatment or adjusting may comprise adding or using a steroid in addition to or in place of another ALN treatment, or a switch in immunosuppressive agent.

In some embodiments of any method herein disclosed involving an ALN treatment or adjusted treatment, whether a subject or patient has received the treatment, or the treatment is selected, administered, or otherwise, mycophenolate may be administered in combination with tacrolimus, voclosporin or belimumab. In these embodiments, mycophenolate may be administered as mycophenolate mofetil or mycophenolic acid.

In another aspect, methods of treating active Lupus Nephritis (ALN) as herein disclosed comprise adjusting an ALN treatment in a subject having increased or comparable measured levels of a plurality of target biomarkers relative to a baseline level and/or control in one or more test urine samples, or alternatively, continuing administration of the treatment in a subject that has decreased measured levels of a plurality of target biomarkers relative to a baseline level and/or control in one or more test urine samples, wherein the target biomarkers comprise Adiponectin, MCP-1, sVCAM-1, and PF4, and none, one or both of vWF and IL-15 and wherein the measured levels are from samples taken within about 6 to about 24 months, preferably 12 months, from onset of the treatment. In these embodiments, the treatment adjusting may comprise switching treatments, wherein administration of a first ALN treatment to the subject is discontinued and instead administration of a second ALN treatment to the subject is initiated, wherein the first and second treatments are distinct selections for example from azathioprine, cyclophosphamide and mycophenolate. For example, a first treatment may comprise mycophenolate and the second treatment may comprise either azathioprine or cyclophosphamide. The second treatment may also comprise addition of a therapeutic agent such as tacrolimus, voclosporin or belimumab.

In some embodiments of any herein disclosed method, test samples from a subject may be acquired, or measured target biomarker levels are from samples taken, within about 6 to 9 months, about 9 to 12 months, about 12 to 18 months, or about 18 to 24 months from onset of the treatment. In other embodiments, the measured levels are from samples taken within about 6 months, about 9 months, about 12 months, about 18 months or about 24 months from onset of the treatment or a LN flare. In some embodiments, the measured levels are from samples taken within about 12±3 months, 9 to 15 months, 24±3 months or within about 21 to 27 months from onset of the treatment or a LN flare.

In another aspect, methods of predicting a treatment outcome in a subject that has active Lupus Nephritis (ALN) comprises the following steps: i) acquiring one or more test urine samples from the subject, wherein the test samples are acquired within about 24±3 months or within about 21 to 27 months following onset of administration of an ALN treatment optionally comprising azathioprine, cyclophosphamide or mycophenolate; ii) measuring levels of a plurality of target biomarkers in the test samples, comprising contacting the test samples with a urinary biomarker panel comprising a plurality of biomarker detection agents, optionally the urinary biomarker panel described herein, wherein each biomarker detection agent is specific for a corresponding target biomarker and the plurality of target biomarkers comprise Adiponectin, MCP-1, sVCAM-1, and PF4, and none, one or both of vWF and IL-15, and optionally one or more control biomarkers; iii) comparing the measured levels of each of the test sample target biomarkers to a baseline level and/or control; and iv) predicting a probability that the subject will experience a subsequent ALN flare within about 6 to about 12 months of acquiring the test samples, wherein the subject has an increased probability of a subsequent ALN flare if at least two of the target biomarkers are Adiponectin, MCP-1, sVCAM-1 and/or PF4 and the measured levels of the said at least two biomarkers reach the cut-off levels relative to a subject having levels of the target biomarkers that are less than the cut-off levels.

In another aspect, methods of treating active Lupus Nephritis (ALN) as herein disclosed comprise administering an ALN treatment optionally comprising azathioprine, cyclophosphamide or mycophenolate to a subject having provided test urine samples wherein measured levels of at least two target biomarkers reach a cut-off level for each of the said target biomarkers, wherein the target biomarkers comprise Adiponectin, MCP-1, sVCAM-1 and/or PF4, and none, one or both of vWF and IL-15.

In some embodiments, in methods as herein described, test samples are taken at about 12 months from onset of an ALN treatment.

In another aspect, methods of monitoring and/or treating a subject with active Lupus Nephritis (ALN) comprise the following steps: i) acquiring one or more test urine samples from the subject; ii) measuring levels of a plurality of target biomarkers in the test samples, comprising contacting the test samples with a urinary biomarker panel comprising a plurality of biomarker detection agents, optionally a urinary biomarker panel as herein described, wherein each biomarker detection agent is specific for a corresponding target biomarker and the plurality of target biomarkers comprise Adiponectin, MCP-1, sVCAM-1, and PF4, and none, one or both of vWF and IL-15, and optionally one or more control biomarkers; iii) comparing the measured levels of each of the test sample target biomarkers to levels of the target biomarkers in one or more control samples, optionally wherein the control samples are baseline samples acquired from the subject or are acquired from one or more healthy control subjects; and iv) administering an ALN treatment based on the measured levels of the target biomarkers in the test samples, wherein at least two of the target biomarkers are Adiponectin, MCP-1, sVCAM-1 and/or PF4 and the measured levels for the said target biomarkers reach a cut-off level for each target biomarker, optionally wherein the treatment comprises administering azathioprine, cyclophosphamide or mycophenolate, or alternatively, adjusting treatments. Adjusting treatment may include discontinuing administration of a first ALN treatment and administering a second ALN treatment, optionally wherein the first and second treatments are distinct selections from azathioprine, cyclophosphamide and mycophenolate or distinct dosages. One or more therapeutic agents may also be added when a method described herein suggests that the subject is not responding sufficiently to the treatment being administered. In some embodiments, the method comprises monitoring the subject, for example if the subject appears to be responding, or is very close to the selected cut-offs for one or more of the target biomarkers described herein.

In some embodiments, monitoring the subject in methods of monitoring and/or treating a subject a subject with active Lupus Nephritis (ALN) may comprise acquiring recall or further samples and carrying out steps (ii) to (iv) with the recall or further samples. In further embodiments, these methods may further comprise identifying individuals at high risk for subsequent LN flares and/or prophylactically treating a future LN flare. For example the identifying can comprise providing the subject with an update, optionally digitally or in person.

Possible early detection of target biomarker levels indicating ALN using the herein disclosed methods and possible early treatment intervention can be advantageous. For example the time from renal flare onset to response to an ALN treatment is a predictor of the extent of renal damage caused by the renal flare. Accordingly, early assessment of likely response or lack of response allows for treatment modification and improved outcomes.

In another aspect, methods of selecting a treatment for Lupus Nephritis (ALN), as herein described comprise at least the following steps: i) acquiring one or more test urine samples; ii) measuring levels of a plurality of target biomarkers in the test samples, comprising contacting the test samples with a biomarker panel as herein described; iii) comparing the measured levels of the target biomarkers in the test samples to levels of the target biomarkers in one or more control samples; and iv) selecting an ALN treatment based on the measured levels of the target biomarkers in the test sample, wherein at least two of the said target biomarkers are Adiponectin, MCP-1, sVCAM-1 and/or PF4 and the measured levels for the said target biomarkers reach a cut-off level for each target biomarker; wherein the treatment comprises for example azathioprine, cyclophosphamide or mycophenolate.

Cut-off levels and/or combinations of the target biomarkers can be used with any of the methods described herein.

In some embodiments, in methods as herein described, the cut-off level for each target biomarker is determined using a binary partitioning method.

In some embodiments, in methods as herein described, each target biomarker cut-off level depends on a selected sensitivity, specificity, positive predictive value (PPV), negative predictive value (NPV), positive likelihood ratio (+LR) and/or negative likelihood ratio (−LR). In other some embodiments, the selected specificity, and/or NPV for each target biomarker is above about 90% or about 95%. In other embodiments, the cut-off levels are based on a selected specificity and PPV above about 95% or about 100%.

In other embodiments, in methods as herein described, the ALN is proliferative Lupus nephritis (PLN).

In still further embodiments, methods as herein described further comprise measuring one or more ALN clinical markers known in the art, for example, serum creatinine, eGFR and/or proteinuria.

In some embodiments, the method further comprises ruling out that the subject has a urinary tract infection. For example the method can comprise performing an assay to detect a urinary tract infection. Where a urinary tract infection is detected, the subject may be recalled for further testing once the urinary tract infection is resolved and before taking further steps, e.g. adjusting or starting treatment, predicting or identifying ALN etc.

In some embodiments, measuring target biomarker levels is effected using an immunoassay such as a multiplex assay system and/or an enzyme-linked immunosorbent assay (ELISA). Other immunological and non-immunological methods can also be used, including for example liquid chromatography-mass spectroscopy, fluorescence spectroscopy, IR and Raman spectroscopies. Methods such as those described in Aitekenov et al, 2021 can also be used (26).

In other embodiments, in methods as herein described, the biomarker detection agents are coupled to a solid support, which may be any solid means known in the art on which a biomarker detection agent, for example an antibody or antibody binding fragment thereof, can be coated or affixed. In some embodiments, the solid support is one or more beads, which may each be uniquely coded, for example, colour-coded with a fluorescent dye or pigment for identification of the biomarker detecting agent it is affixed to and target biomarker that specifically associates with the biomarker detecting agent. In other embodiments, the solid support is a well plate configured to bind to or be coated with a biomarker detection agent, or a chip configured to bind to or be coated with a biomarker detection agent, for example an electronic device configured for detecting and/or measuring light or electrochemiluminescence signals.

Uses of each of the methods are also contemplated.

While the present disclosure has been described with reference to what are presently considered to be the preferred examples, it is to be understood that the application is not limited to the disclosed examples. To the contrary, the application is intended to cover various modifications and equivalent arrangements included within the spirit and scope of the appended claims.

### EXAMPLES

Design and Patients: Patients with Systemic Lupus Erythematosus (SLE) from the University of Toronto Lupus cohort and the LuNNET cohort (16, 25) were included in the following Examples. All patients met the revised 1997 ACR classification criteria for SLE (26) or had three criteria and a supportive biopsy (skin or kidney).

Urinary Biomarker Assays: In the following Examples, all urine samples were spun to remove cellular debris and frozen at −80° C. To avoid repeated freeze/thaws, samples were thawed once on ice, sub-aliquoted, re-frozen at −80° C., and then individual aliquots thawed immediately prior to use. For the first stage of the study, the urinary concentrations of 15 analytes (Clusterin, Cystatin C, NGAL, PF4, vWF, sVCAM-1, GM-CSF, GRO, IL-15, IL-6, MCP-1, Adiponectin, PAI-1, MMP-7 and TIMP-1) were measured by coupled bead assay (Luminex using MILLIPLEX® Map Kits (EMC Millipore Corporation) through Eve Technologies Inc.). Further information regarding the sensitivity and dynamic range of the assays can be found on the company website. For the majority of assays, the urine samples were run undiluted except for Clusterin and Cystatin C, which were diluted 1/50 and TIMP-1, which was diluted 1/5. All analytes were measured in duplicate, with a single sample on each of two separate plates and averaged. Urinary biomarker levels were considered abnormal if they were >2 SD above the mean of the 24 healthy controls.

Statistical Analysis: In the following Examples, descriptive statistics were generated for patients' baseline characteristics for the two cohorts, with baseline categorical variables being presented as counts and percentages. Continuous biomarker variables are presented as median and IQR or mean and standard deviation, as appropriate.

### Example 1

### Materials and Methods

In Example 1, Luminex was used to examine whether the 15 urinary biomarkers could predict treatment response. For this stage, the cohort was composed of SLE patients from the LuNNET cohort, recruited from April 2006 to December 2011, all of whom had biopsy proven ALN. The KB was performed±3 months from the baseline urine sample collection, with all patients being followed longitudinally for a minimum of 2 years at the University of Toronto Lupus Clinic. Follow-up urine samples were collected every 3-6 months up to 24±3 months.

The response to treatment was established after 24 months of follow-up, using the following criteria: 1) Complete response (CR): reduction in a 24 hour protein excretion to <500 mg/day with normal serum creatinine or serum creatinine within 15% of previous baseline; 2) Partial response (PR): >50% reduction in proteinuria and to non-nephrotic levels, with serum creatinine within 25% of previous baseline; and 3) No response (NR): patients who did not achieve CR or PR (2, 27). Samples from 24 healthy controls were also assayed to enable determination of normal biomarker values.

### Statistical Analysis

Logistic regression models were used to determine if the baseline urinary biomarker levels, or the absolute or percentage decrease, after 12 months of therapy predicted CR to treatment at 24 months. For this analysis non-CR (PR and NR) were pooled together. A scatter plot of each of the patient measurements at different time points, as well as a smooth line were plotted to visualize the trend of the curve depending on the response (CR vs PR/NR).

### Results

Only a subset of urinary biomarkers demonstrated change over time that associated with treatment response.

21 biopsy-proven ALN patients were included, 19 (90.5%) of whom had proliferative LN (see Table 1). The mean age at baseline was 32.15 years and 85.7% of patients were female. The predominant ethnicity was Caucasian (47.6%), followed by Asian (23.8%) and Afro-Caribbean (14.3%). The mean SLE disease duration was 3.69 years and the average time since the start of the LN flare to the urine sample collection was 1.19±1.12 months. Twelve (57.1%), 4 (19.1%), and 5 (23.8%) patients had a complete (CR), partial (PR) and no remission (NR), respectively after 24 months of conventional therapy.

TABLE 1
Baseline demographic and clinical characteristics of the patient cohorts^{†}.

| | Example 1 | | Example 2 cross-sectional cohort N = 247 | | | | | |
|---|---|---|---|---|---|---|---|---|
| | ALN (N = 21) | | ALN (N = 24) | | RLN (N = 79) | | NLN (N = 144) | |
| Ethnicity, n (%) | | | | | | | | |
| Caucasian | 10 | (47.6) | 8 | (33.3) | 41 | (51.8) | 82 | (56.9) |
| Afro-Caribbean | 3 | (14.3) | 9 | (37.5) | 14 | (17.7) | 33 | (23.1) |
| Asian | 5 | (23.8) | 4 | (16.6) | 11 | (13.9) | 12 | (8.4) |
| Other | 3 | (14.3) | 3 | (12.5) | 13 | (16.5) | 17 | (11.9) |
| Female, n (%) | 18 | (85.7) | 20 | (83.3) | 64 | (81.0) | 132 | (91.6) |
| Age (years), Median (IQR) | 28.90 | (23.5-44.0) | 28.6 | (24.6-36.1) | 41 | (28.9-52.4) | 38 | (29.6-52.7) |
| Duration SLE (years), | 2.90 | (0.1-7.5) | 7.7 | (3.57-10.19) | 9.27 | (4.20-17.46) | 7.06 | (2.83-13.52) |
| Median (IQR) | | | | | | | | |
| Time from LN flare (months)*, | 1.0 | (0-2.0) | 5.5 | (2.7-10) | 48 | (24-108) | NA | |
| Median (IQR) | | | | | | | | |
| Antiphospholopid | 1 | (4.76) | 1 | (4.16) | 9 | (11.39) | 9 | (6.25) |
| síndrome, n (%) | | | | | | | | |
| Hypertension, n (%) | 5 | (23.80) | 6 | (25.00) | 22 | (27.84) | 19 | (13.19) |
| Diabetes Mellitus, n (%) | 0 | (0) | 0 | (0) | 6 | (7.59) | 3 | (2.08) |
| Clinical features, n (%) | | | | | | | | |
| Mucocutaneous | 7 | (33.33) | 4 | (16.66) | 8 | (10.12) | 13 | (9.02) |
| Musculoskeletal | 8 | (38.09) | 0 | (0) | 2 | (2.53) | 7 | (4.86) |
| Serositis | 2 | (9.52) | 0 | (0) | 0 | (0) | 1 | (0.69) |
| Hematologic | 3 | (14.28) | 1 | (4.16) | 6 | (7.59) | 13 | (9.02) |
| Central Nervous System | 0 | (0) | 0 | (0) | 1 | (1.26) | 1 | (0.69) |
| Vasculitis | 1 | (4.76) | 0 | (0) | 1 | (1.26) | 0 | (0) |
| Renal | 21 | (100.00) | 24 | (100.00) | 0 | (0) | 0 | (0) |
| Fever | 2 | (9.52) | 0 | (0) | 1 | (1.26) | 0 | (0) |
| SLEDAI, total score, | 18.0 | (14.0-24.0) | 10.0 | (6.0-13) | 30 | (0-4.0) | 2.0 | (0-4.0) |
| Median (IQR) | | | | | | | | |
| SLEDAI, renal, | 12.0 | (8.0-12.0) | 4.0 | (4-8) | — | | — | |
| Median (IQR) | | | | | | | | |
| Anti-dsDNA Ab (IU/ml), | 100.0 | (19.0-101.0) | 55 | (24-254) | 15 | (1-55) | 3 | (1-15) |
| Median (IQR) | | | | | | | | |
| Positive | 4 | (19.04) | 4 | (16.66) | 18 | (22.78) | 33 | (22.91) |
| Antiphospholipid | | | | | | | | |
| Abs, n (%) | | | | | | | | |
| C3, g/rL, Median (IQR) | 0.62 | (0.35-0.77) | 0.86 | (0.67-0.99) | 0.99 | (0.82-1.12) | 1.04 | (0.85-1.22) |
| C4, gr/L, Median (IQR) | 0.07 | (0.05-0.14) | 0.14 | (0.13-0.20) | 0.17 | (0.13-0.23) | 0.19 | (0.14-0.25) |
| Serum Albumin (gr/L), | 29.0 | (22.0-32.0) | 34.0 | (31.5-37.5) | 41.0 | (38.0-43.0) | 42.0 | (39.0-44.0) |
| Median (IQR) | | | | | | | | |
| Serum Creatinine (umol/L), | 89.0 | (71.0-134.0) | 77.5 | (66.5-100) | 77.0 | (57.0-81.0) | 64.5 | (56.5-74.5) |
| Median (IQR) | | | | | | | | |
| GFR <60 ml/min/m2, n (%) | 6 | (28.6) | 6 | (25.0) | 11 | (13.9) | 0 | (0) |
| GFR <30 ml/min/m2, n (%) | 3 | (14.3) | 2 | (8.3) | 3 | (3.8) | 0 | (0) |
| GFR <15 ml/min/m2 or RRT, n (%) | 0 | (0) | 0 | (0) | 0 | | 0 | (0) |
| 24-hour Protein excretion (gr), | 2.05 | (1.52-3.82) | 1.13 | (0.70-2.65) | 0 | (0-0.4) | 0 | (0-0.4) |
| Median (IQR) | | | | | | | | |
| Kidney biopsy Class, n (%) | 21 | (100) | 11 | (45.8) ^{#} | | | | |
| I | 0 | (0) | 0 | (0) | | | | |
| II | 0 | (0) | 0 | (0) | | | | |
| III | 1 | (4.8) | 2 | (0.08) | | | | |
| IV | 10 | (47.6) | 5 | (0.2) | | | | |
| V | 2 | (9.5) | 1 | (0.04) | | | | |
| III + V | 4 | (19) | 1 | (0.04) | | | | |
| IV + V | 4 | (19) | 2 | (0.08) | | | | |
| VI | 0 | (0) | 0 | (0) | | | | |
| Activity Index, Median (IQR) | 11.0 | (6.0-13.0) | 7.0 | (2.25-9.75) | | | | |
| Chronicity Index, Median (IQR) | 3.0 | (2.0-4.0) | 3.0 | (2.25-4.75) | | | | |
| Prednisone, n (%) | 21 | (100) | 22 | (91.7) | 47 | (59.5) | 71 | (49.3) |
| Prednisone dose (mg), | 45.0 | (40.0-50.0) | 15.0 | (10.0-20.0) | 5.0 | (5.0-10.0) | 4.0 | (5.0-7.5) |
| Median (IQR) | | | | | | | | |
| Antimalarial, n (%) | 20 | (95.2) | 20 | (83.3) | 67 | (84.8) | 124 | (86.1) |
| Immunosuppressive, n (%) | 21 | (100) | 23 | (95.8) | 56 | (70.8) | 83 | (57.6) |
| Azathioprine, n (%) | 3 | (14.3) | 1 | (4.2) | 11 | (13.9) | 31 | (21.5) |
| Azathioprine dose (mg), | 125.0 | (100.0/150.0) | 100.0 | (100.0/100.0) | 100.0 | (100.0/150.0) | 100.0 | (75.0/150.0) |
| Median (IQR) | | | | | | | | |
| Mychophenolate, n (%) | 16 | (76.2) | 20 | (83.3) | 42 | (53.2) | 37 | (25.7) |
| Mychophenolate dose (gr), | 2.0 | (2.0-3.0) | 2.0 | (2.0-3.0) | 2.0.0 | (1.5-2.5) | 2.0 | (2.0-3.0) |
| Median (IQR) | | | | | | | | |
| Cyclophosphamide, n (%) | 2 | (19.5) | 0 | (0) | 0 | (0) | 0 | (0) |

| | | | | | | | |
|---|---|---|---|---|---|---|---|
| Methotrexate, n (%) | — | 1 | (4.2) | 3 | (3.8) | 15 | (10.4) |
| Methotrexate dose (mg), | — | 22.5 | (20.0-25.0) | 12.5 | (10.0-15.0) | 17.5 | (15.0-20.0) |
| Median (IQR) | | | | | | | |

^{†}Baseline clinical characteristics are at the time of the urine sample collection,
*Time from LN flare to urine sample collection (months),
^{#} Remaining 13 patients did not have a KB at the time of the urine sample collection, of whom 3 did not have a prior KB and 10 had a prior KB (9 class III or IV with or without class V and 1 pure class V).
GFR = Glomerular Filtration Rate, RRT = Renal Replacement Therapy.

Patients who achieved CR, PR and NR were treated similarly. The dose of prednisone used at baseline was similar for the 3 groups (45, 52 and 39 mg for CR, PR and NR, respectively, p=0.241). In the CR group 2 (16%) were treated with Azathioprine, 2 (16%) with Cyclophosphamide and 8 (66.6%) with Mycophenolate. All patients with PR and 4 (80%) of the patients with NR were treated with Mycophenolate. The remaining NR patient was treated with Azathioprine.

The baseline levels of urinary biomarkers did not predict response to therapy at 24 months. However, the percentage decrease in Adiponectin, MCP-1, sVCAM-1, PF4, IL-15 and vWF at 12±3 months predicted response to therapy at 24 months. (FIG. 1 and Table 2).

TABLE 2
Logistic regression analysis assessing baseline levels and
percentage decrease at 12 ± 3 months as predictors of
complete response at month 24 for the urinary biomarkers. N = 21

| | Baseline | | % Decrease at month 12 | | |
|---|---|---|---|---|---|
| Biomarkers | OR (95% CI) | P value | OR (95% CI) | | P value |
| Adiponectin | 1.00 (1.00-1.00) | 0.58 | NA¹ | | NA¹ |
| MCP-1 | 1.00 (1.00-1.00) | 0.79 | NA² | | NA² |
| sVCAM | 1.00 (1.00-1.00) | 0.53 | 0.05 | (0.006-0.44) | 0.007 |
| PF4 | 0.97 (0.84-1.13) | 0.71 | 0.042 | (0.004-0.49) | 0.011 |
| vWF | 1.02 (0.96-1.08) | 0.56 | 0.045 | (0.004-0.54) | 0.014 |
| IL15 | 1.09 (0.89-1.34) | 0.41 | 0.143 | (0.02-0.93) | 0.042 |
| Cystatin-C | 0.99 (0.97-1.00) | 0.21 | 1.00 | (1.00-1.00) | 0.94 |
| PAI-1 | 0.99 (0.99-1.00) | 0.23 | 0.98 | (0.96-1.00) | 0.13 |
| GM-CSF | 1.29 (0.83-1.98) | 0.25 | 1.00 | (1.00-1.00) | 0.52 |
| Lipocalin | 0.99 (0.99-1.00) | 0.52 | 1.00 | (1.00-1.00) | 0.87 |
| GRO | 0.99 (0.99-1.00) | 0.69 | 0.99 | (0.99-1.00) | 0.36 |
| MMP7 | 1.00 (1.00-1.00) | 0.72 | 0.99 | (0.99-1.00) | 0.86 |
| IL6 | 0.99 (0.97-1.02) | 0.86 | 0.99 | (0.99-1.00) | 0.73 |
| Clusterin | 1.00 (1.00-1.00) | 0.90 | 1.00 | (0.99-1.00) | 0.94 |
| TIMP-1 | 0.99 (0.98-1.01) | 0.92 | 0.99 | (0.99-1.00) | 0.14 |

NA¹—Not Applicable due to perfect specificity.
NA²—Not Applicable due to perfect sensitivity.

### Example 2

### Materials and Methods

In Example 2, the most predictive urinary biomarkers for response to treatment that were identified in Example 1 (Adiponectin, MCP-1, sVCAM, PF4, IL-15 and vWF) were assayed using ELISA, to further validate their ability to accurately detect ALN patients. For this second Example, a larger cross-sectional cohort was acquired. SLE patients from the University of Toronto Lupus cohort (enrolled within the last 5 years, to assure no overlap with the LuNNET cohort) were consecutively recruited from July 2016 to March 2019, when attending their scheduled clinic appointment. For this cohort, ALN was defined clinically as a LN flare that occurred within the last 12 months from the urine collection, with a 24 hour urinary protein excretion of ≥500 mg/day, which was interpreted by the physician in charge as being secondary to active renal inflammation prompting a change in immunosuppressive therapy. Non-ALN patients were divided into two groups: 1) Patients with RLN, defined as the presence of a history of LN but no clinical signs of renal activity at the time of sample collection, with a 24 hour urinary protein excretion of <500 mg/day or the presence of chronic proteinuria which was interpreted by the physician in charge as being secondary to damage and not requiring a change in immunosuppressive therapy. Chronic proteinuria was defined as stable proteinuria present for at least 1 year, in the presence of chronic kidney disease (CKD, defined as a eGFR<60 ml/min/m2) and/or other comorbidities known to cause of proteinuria, such as diabetes mellitus and hypertension; and 2) NLN patients, with no history of LN and no clinical signs of ALN (urinary protein excretion <500 mg/day) at the time of the urine sampling, but who could have extra-renal SLE activity.

sVCAM-1 (Cat #DY809), MCP-1 (Cat #DY279), Adiponectin (Cat #DY1065), PF-4 (Cat #DY795), vWF (Cat #DY2764-05) and IL-15 (Cat #DY247) were measured by ELISA, using Duoset and Ancillary Reagent Kits (Cat #DY008) obtained from R&D Systems, and processed following the manufacturer's protocols. Optimal dilutions for each cytokine ELISA were determined in preliminary experiments and were 1/16 for Adiponectin, 1/128 for SVCAM-1, 1/8 for MCP-1, and 1/4 for PF-4, vWF, and IL-15. For the majority of samples IL-15 and vWF concentrations were below the limit of detection and therefore were not pursued further. All samples were run in duplicate, averaged, and their cytokine concentration computed from a In-In plot of the cytokine standard curve, with adjustment for the dilution factor. Any samples with raw absorbance values that were under the lower limit of the standard curve using the optimal dilution, were re-run at lower dilutions and those that were below the standard curve at a 1/4 dilution were given the lowest standard curve value for ensuing calculations.

### Statistical Analysis

The Kruskal-Wallis test was used to assess the differences in biomarker measures between groups. Logistic regression models were used to assess the impact of each of the potential continuous predictors to discriminate between ALN and non-ALN. A binary partitioning method was used to obtain the optimum cut-off for each biomarker that discriminated between ALN and non-ALN (RLN and NLN). Receiver Operating Characteristic curves were generated for each individual biomarker.

Sensitivity, specificity, positive predictive value (PPV), negative predictive value (NPV), positive likelihood ratio (+LR) and negative likelihood ratios (−LR) with 95% confidence intervals were calculated to determine the accuracy of detecting active LN when: 1 or more, 2 or more or 3 or more biomarkers were elevated. Sensitivity, specificity, PPV and NPV above 80% were considered good and above 90% excellent. Likelihood ratios above 10 for +LR and below 0.1 for −LR were considered to provide strong evidence to rule in or rule out diagnoses (25).

### Results

The most predictive biomarkers in a cross-sectional SLE cohort were validated. A total of 247 SLE patients from the University of Toronto Lupus Clinic were included in Example 2, of whom 24 (9.7%) had ALN, 79 (31.9%) had RLN and 144 (58.3%) had NLN patients. All ALN patients were within 12 months of detection of the LN flare, with a mean time of 6 months between the initiation of the flare and the urine collection. Since our criteria for including ALN were clinical, only 11 (45.8%) had a KB at the time of their LN flare, of whom 10 (41.7%) had a proliferative class, either Ill or IV with or without class V, and 1 (0.04%) had pure membranous class V. The remaining 13 patients did not have a KB performed at the time of the flare. However, 10 had a prior KB (9 class Ill or IV with or without class V, and 1 pure class V). Table 1 shows the baseline demographic and clinical characteristics of the cohort.

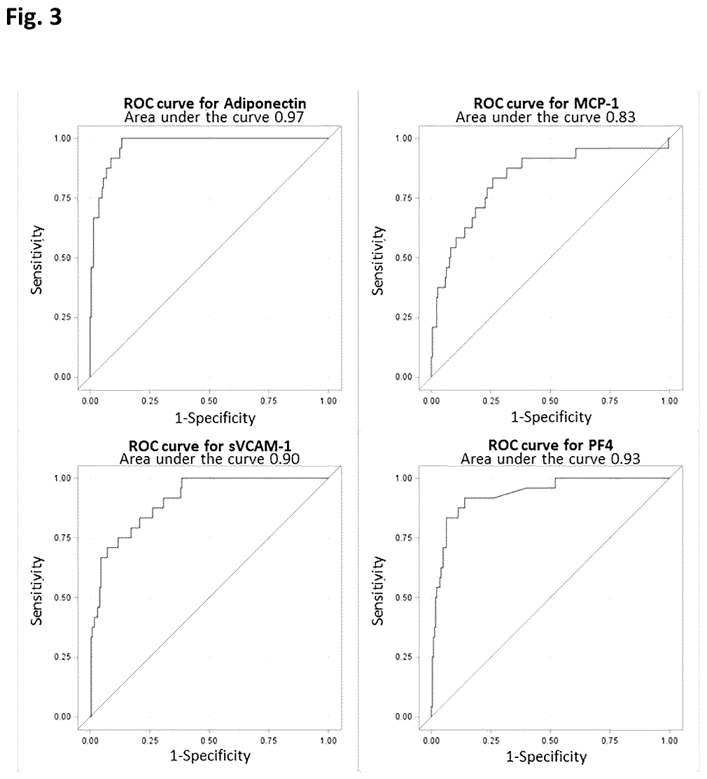

Based on the findings from Example 1, 6 urinary biomarkers were measured by ELISA in the cross-sectional cohort, including Adiponectin, MCP-1, sVCAM-1, PF4, vWF and IL-15. Patients with ALN had higher levels of all 4 remaining analytes, including Adiponectin, MCP-1, sVCAM-1 and PF4, in comparison to patients with RLN and NLN, as shown in FIG. 2 and Table 3. Cut-offs with the best operating characteristics to detect ALN patients were produced for each biomarker (Adiponectin 18000 pg/ml, MCP-1 1341 pg/ml, SVCAM-1 46000 pg/ml and PF4 134 pg/ml), see FIG. 3 for Receiver Operating Characteristic curves. Adiponectin was the most sensitive, with a high NPV (99%) and good −LR (0.09). However, 2 patients that were classified as ALN did not meet the Adiponectin cut-off. These patients were in the 4th and 10th month of the onset of their LN flare. In addition, Adiponectin alone had a low PPV (52%). In contrast, MCP-1 and PF4 had high specificities, but low PPV's (Table 4).

TABLE 3
Distribution of Biomarkers depending on their activity status:
ALN, RLN and NLN. Data expressed as Median (IQR).

| | ALN (N = 24) | RLN (N = 79) | NLN (N = 144) | P-value |
|---|---|---|---|---|
| Adiponectin | 46888.3 | 5183.0 | 3566.4 | <.0001 |
| pg/ml | (24387.8, | (2285.6, | (1329.7, | |
| | 78227.1) | 10900.7) | 8377.3) | |
| PF4 | 321.7 | 35.3 | 30.3 | <.0001 |
| pg/ml | (138.0, | (26.1, | (26.6, | |
| | 1547.2) | 44.2) | 39.4) | |
| MCP-1 | 771.7 | 125.6 | 100.0 | <.0001 |
| pg/ml | (291.4, | (50.5, | (47.8, | |
| | 1780.7) | 300.3) | 236.0) | |
| sVCAM-1 | 125754.7 | 18383.7 | 9530.2 | <.0001 |
| pg/ml | (62505.9, | (7046.1, | (2636.6, | |
| | 268615.7) | 46158.2) | 25781.2) | |

TABLE 4
Operating characteristics for individual biomarkers

| | Sensitivity | Specificity | PPV | NPV | +LR | −LR |
|---|---|---|---|---|---|---|
| Biomarkers | (95% CI) | (95% CI) | (95% CI) | (95% CI) | (95% CI) | (95% CI) |
| Adiponectin (18000) | 91.7 | 90.9 | 52.4 | 99.0 | 10 | 0.09 |
| | (73.0-99.0) | (86.3-94.3) | (36.4-68.0) | (96.5-99.9 | (6.50-16) | (0.02-0.35) |
| MCP-1 (1341) | 37.5 | 97.3 | 60.0 | 93.5 | 14 | 0.64 |
| | (18.8-59.4) | (94.2-99.0) | (32.3-83.7) | (89.5-96.3) | (5.40-36) | (0.47-0.88) |
| PF4 (134) | 83.3 | 93.7 | 58.8 | 98.1 | 13 | 0.18 |
| | (62.6-95.3) | (89.7-96.5) | (40.7-75.4) | (95.2-99.5) | (7.72-23) | (0.07-0.44) |
| sVCAM-1 (46000) | 79.2 | 81.1 | 31.2 | 97.3 | 4.2 | 0.26 |
| | (57.9-92.9) | (75.3-86.0) | (19.9-44.3) | (93.8-99.1) | (2.9-5.9) | (0.12-0.56) |
| sVCAM-1 (103700) | 66.7 | 95.5 | 61.5 | 96.4 | 15 | 0.35 |
| | (44.7-84.4) | (91.9-97.8) | (40.6-79.8) | (93.0-98.4) | (7.6-29) | (0.20-0.62) |

PPV = Positive Predictive Value, NPV = Negative Predictive Value, +LR = Positive likelihood ratio, −LR = Negative likelihood ratio.

### Example 3

### Materials and Methods

In Example 3, the sensitivity of urinary biomarker cut-offs, established in the cross-sectional cohort of Example 2, to identify ALN patients was validated, and determined if they operated similarly for proliferative and non-proliferative ALN classes. For this Example, urinary Adiponectin, MCP-1, sVCAM and PF4 were measured using ELISA. All patients had biopsy proven ALN±3 months from the urine sample collection.

sVCAM-1 (Cat #DY809), MCP-1 (Cat #DY279), Adiponectin (Cat #DY1065), PF-4 (Cat #DY795), vWF (Cat #DY2764-05) and IL-15 (Cat #DY247) were measured by ELISA, using Duoset and Ancillary Reagent Kits (Cat #DY008) obtained from R&D Systems, and processed following the manufacturer's protocols. Optimal dilutions for each cytokine ELISA were determined in preliminary experiments and were 1/16 for Adiponectin, 1/128 for SVCAM-1, 1/8 for MCP-1, and 1/4 for PF-4, vWF, and IL-15. For the majority of samples IL-15 and vWF concentrations were below the limit of detection and therefore were not pursued further. All samples were run in duplicate, averaged, and their cytokine concentration computed from a In-In plot of the cytokine standard curve, with adjustment for the dilution factor. Any samples with raw absorbance values that were under the lower limit of the standard curve using the optimal dilution, were re-run at lower dilutions and those that were below the standard curve at a 1/4 dilution were given the lowest standard curve value for ensuing calculations.

### Statistical Analysis

Sensitivity with 95% confidence intervals was calculated for the presence of 2 or more, or 3 or more elevated biomarkers. All p-values were 2-sided and for the statistical analyses, a p<0.05 was considered to indicate a statistically significant result. Statistical analysis was performed using version 9.4 of the SAS system for Windows, Copyright 2002-2012 SAS Institute, Inc., Cary, NC.

Sensitivity, specificity, positive predictive value (PPV), negative predictive value (NPV), positive likelihood ratio (+LR) and negative likelihood ratios (−LR) with 95% confidence intervals were calculated to determine the accuracy of detecting active LN when: 1 or more, 2 or more or 3 or more biomarkers were elevated. Sensitivity, specificity, PPV and NPV above 80% were considered good and above 90% excellent. Likelihood ratios above 10 for +LR and below 0.1 for −LR were considered to provide strong evidence to rule in or rule out diagnoses (28).

### Results

The optimal biomarker combination to accurately detect ALN was identified. Given that none of the 4 urinary biomarkers by themselves had excellent operating characteristics, whether different combinations and/or numbers of elevated biomarkers could more accurately identify ALN patients (Table 5) was analyzed. The operating characteristics for any combination of 2 elevated biomarkers were good, with a sensitivity and specificity above 90%, high NPV (99%) and excellent −LR (0.09). These results were similar to those for Adiponectin alone.

TABLE 5
Operating characteristics for different combinations and number
of elevated urinary biomarkers to accurately detect ALN patients

| | Operating characteristics calculated using sVCAM cutt-off of 46000 | | | | | |
|---|---|---|---|---|---|---|
| | Sensitivity | Specificity | PPV | NPV | +LR | −LR |
| Biomarkers | (95% CI) | (95% CI) | (95% CI) | (95% CI) | (95% CI) | (95% CI) |
| 2 Elevated biomarkers* | 91.7 (73.0- | 90.1 (85.4- | 50.0 (34.6- | 99.0 (96.5- | 9.25 | 0.09 (0.02- |
| | 99.0) | 93.7) | 65.4) | 99.9) | (6.11-14) | 0.35) |
| Different combinations | | | | | | |
| Adiponectin-MCP-1 | 33.3 (15.6- | 100 (98.4- | 100 (63.1- | 93.3 (89.3- | NA | 0.67 (0.50- |
| | 55.3) | 100) | 100) | 96.1) | 28 (12-63) | 0.88) |
| Adiponectin-PF4 | 75.0 (53.3- | 97.3 (94.2- | 75.0 (53.3- | 97.3 (94.2- | NA | 0.26 (0.13- |
| | 90.2) | 99.0) | 90.2) | 99.0) | | 0.51) |
| MCP-1-PF4 | 37.5 (18.8- | 100 (98.4- | 100 (66.4- | 93.7 (89.8- | 10 (6.04- | 0.62 (0.46- |
| | 59.4) | 100) | 100) | 96.4) | 4.18) | 0.84) |
| sVCAM-1-Adiponectin | 70.8 (48.9- | 93.2 (89.1- | 53.1 (34.7- | 96.7 (93.4- | 37 (8.33- | 0.31 (0.17- |
| | 87.4) | 96.2) | 70.9) | 98.7) | 164) | 0.58) |
| sVCAM-1-MCP-1 | 33.3 (15.6- | 99.1 (96.8- | 80.0 (44.4- | 93.2 (89.2- | 22 (10-49) | 0.67 (0.51- |
| | 55.3) | 99.9) | 97.5) | 96.1) | | 0.89) |
| sVCAM-PF4 | 70.8 48.9- | 96.9 (93.6- | 70.8 (48.9- | 96.9 (93.6- | | 0.30 (0.16- |
| | 87.4) | 98.7) | 97.4) | 98.7) | | 0.56) |
| 3 Elevated biomarkers^{#} | 70.8 (48.9- | 98.2 (95.5- | 81.0 (58.1- | 96.9 (93.7- | 39 (14- | 0.30 (0.16- |
| | 87.4) | 99.5) | 94.6) | 98.7) | 107) | 0.55) |
| Different combinations | | | | | | |
| Adiponectin-MCP-1- | 29.2 (12.6- | 100 (98.4- | 100 (59.0- | 92.9 (88.9- | NA | 0.71 (0.54- |
| sVCAM-1 | 51.1) | 100) | 100) | 95.8) | | 0.91) |
| Adiponectin-MCP-1-PF4 | 33.3 (15.6- | 100 (98.4- | 100 (63.1- | 93.3 (89.3- | NA | 0.67 (0.50- |
| | 55.3) | 100) | 100) | 96.1) | | 0.88) |
| MCP-1-sVCAM-1-PF4 | 33.3 (15.6- | 100 (98.4- | 100 (63.1- | 93.3 (89.3- | NA | 0.67 (0.50- |
| | 55.3) | 100) | 100) | 96.1) | | 0.88) |
| | Operating characteristics calculated using sVCAM cut-off of 103700 | | | | | |
| 2 Elevated biomarkers* | 87.5 (67.6- | 94.6 (90.8- | 63.6 (45.1- | 98.6 (95.9- | 16.2 | 0.13 (0.05- |
| | 97.3) | 97.2) | 79.6) | 99.7) | (9.15-29) | 0.38) |
| Different combinations | | | | | | |
| sVCAM-1-Adiponectin | 58.3 (36.6- | 97.8 (94.8- | 73.7 (48.8- | 95.6 (92.1- | 26 (10-66) | 0.43 (0.27- |
| | 44.9) | 99.3) | 90.9) | 97.9) | | 0.68) |
| sVCAM-1-MCP-1 | 33.3 (15.6- | 99.1 (96.8- | 80.0 (44.4- | 93.2 (89.2- | 37 (8.33- | 0.67 (0.51- |
| | 55.3) | 99.9) | 97.5) | 96.1) | 164) | 0.89) |
| sVCAM-1-PF4 | 62.5 (40.6- | 98.7 (96.1- | 89.3 (59.6- | 96.1 (92.6- | 46 (14- | 0.38 (0.23- |
| | 81.2) | 99.7) | 96.4) | 98.2) | 148) | 0.64) |
| 3 Elevated biomarkers^{#} | 62.5 (40.6- | 99.1 (96.8- | 88.2 (63.6- | 96.1 (92.7- | 69 (17- | 0.38 (0.23- |
| | 81.2) | 99.9) | 98.5) | 98.2) | 285) | 0.63) |
| Different combinations | | | | | | |
| Adiponectin-MCP-1- | 29.2 (12.6- | 100 (98.4- | 100 (59.1- | 92.9 (88.9- | NA | 0.71 (0.54- |
| sVCAM-1 | 51.1) | 100) | 100) | 95.8) | | 0.91) |
| MCP-1-sVCAM-1-PF4 | 33.3 (15.6- | 100 (98.4- | 100 (63.1- | 93.3 (89.3- | NA | 0.67 (0.50- |
| | 55.3) | 100) | 100) | 96.1) | | 0.88) |

*Any 2 of the 4 biomarkers elevated,
^{#}Any 3 of the 4 biomarkers elevated.
PPV = Positive Predictive Value, NPV = Negative Predictive Value, +LR = Positive likelihood ratio, −LR = Negative likelihood ratio, NA = Not Applicable due to perfect specificity.

When analyzing the different combinations of 2 elevated biomarkers, the MCP-1-Adiponectin and MCP-1-PF4 combinations had specificities and PPVs of 100%, the combination of Adiponectin-PF4 and MCP-1-sVCAM-1 had a lower PPVs, but still excellent +LRs. sVCAM-1-Adiponectin and sVCAM-1-PF4 combinations had good +LRs but had the lowest PPVs.

Overall, the sensitivities for the individual combinations of 2 biomarkers were all below 80% (Table 4).

Given that the combinations of 2 elevated biomarkers including sVCAM-1 had lower PPV, it was assessed whether increasing the cut-off for sVCAM-1 from 46000 to 103700 improved the operating characteristics. By doing this, the PPV and +LR of the combinations of sVCAM-1-Adiponectin and sVCAM-1-PF4 substantially improved as seen in Table 4. The number of false positives for the presence of any combination of 2 elevated biomarkers decreased from 22 (sVCAM-1 cut-off of 46000) to 12 (sVCAM-1 cut-off of 103700). Of the remaining 12 false positives, 9 had RLN and 3 were NLN patients. From the RLN group, 4 had their last LN flare ≤2 years before the study (1 of which developed a subsequent flare 2 years later), 3 had chronic proteinuria (all with CKD, 1 of which also had type 2 diabetes mellitus), and 1 had an active urinary tract infection which required antibiotic therapy.

The presence of 3 biomarkers above the established cut-off, irrespective of the combination and the cut-off of sVCAM-1, had excellent specificity, PPV and +LR (Table 4).

### Example 4

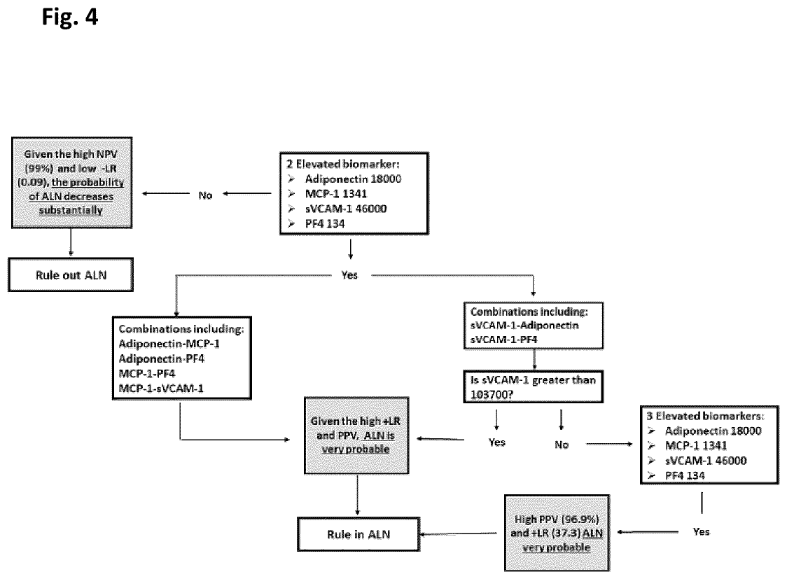

A two-step approach provides the best accuracy for detecting ALN patients (FIG. 4). In the first step, the following “rule out ALN” criteria applies. If there are <2 elevated biomarkers using the lower cut-off for sVCAM-1 (46,000), given the low-LR (0.09) and high NPV (99%), the probability of ALN reduces substantially. For the “rule in ALN” criteria the following approach applies. If 2 biomarkers are elevated including the following combinations Adiponectin-MCP-1, Adiponectin-PF4, MCP-1-PF4 and sVCAM-1-MCP-1, given the PPV and +LR, the diagnosis of LN is very probable. On the other hand, if the combination of 2 elevated biomarkers includes sVCAM-1-Adiponectin and sVCAM-1-PF4, then in order to improve accuracy increasing the cut-off of sVCAM-1 from 46000 to 103700 is preferable. If there are 3 or more elevated biomarkers, irrespective of the combination and sVCAM-1 cutoff, taking into consideration the high PPV (96.9%) and +LR (37.3), LN is very likely. Urinary tract infections should be ruled out, as they may cause false positive results.

“Rule out ALN criteria” at 12 months following ALN flare predicts response to treatment at 24 months. In Example 1, it was determined that the percentage decrease of Adiponectin, MCP-1, sVCAM-1, PF4, vWF and IL-15 after 12±3 months of treatment predicted response to therapy at 24 months. In order to evaluate if the rule out criteria (presence of <2 elevated urinary biomarkers, with sVCAM-1 cut-off of 46,000) could also serve as a predictor of response to treatment a subpopulation of the cross-sectional cohort of Example 2 whose urine sample was collected at 12±3 months after their LN flare was analyzed. Of the twenty-two patients in the analysis, 12 had <2 elevated biomarkers, with 11 achieving a CR at 24 months. In contrast only 4 out of 10 patients with ≥2 elevated biomarkers achieved a CR (p=0.02, Fisher's exact test). The operating characteristics for this subpopulation analysis were as follows: sensitivity (73.3 [95% Cl 44.9-92.2]), specificity (85.7 [95% Cl 42.1-99.6]), PPV (91.7 [95% Cl 61.5-99.8]) and NPV (60.0 [95% Cl 26.2-87.8]).

The “Rule in ALN” criteria operate for proliferative ALN: To corroborate the sensitivity of the “rule in ALN” criteria established in the cross-sectional cohort of Example 2 and determine if they operate similarly for proliferative and non-proliferative LN classes, urinary Adiponectin, MCP-1, sVCAM and PF4 were measured in a biopsy-proven ALN cohort. A total of 53 patients were included, of whom 35 had proliferative LN and 18 non-proliferative class (4 with class V and chronic proliferative LN and 14 with pure II or V LN classes). Table 6 shows their demographic and clinical characteristics at the time of the urine collection. As seen in Table 7, the sensitivity of the “rule in ALN” criteria was similar for the group of proliferative ALN, 91.4% for the presence of 2 or more elevated biomarkers (higher sVCAM cut-off of 103,700) and 77.1% for the presence of 3 or more elevated biomarkers (sVCAM cut-off of 46,000).

TABLE 6
Baseline demographic and clinical characteristics
of the biopsy-proven ALN cohort, N = 53.

| Ethnicity, n (%) | | |
|---|---|---|
| Caucasian | 23 | (43.4) |
| Afro-Caribbean | 13 | (24.5) |
| Asian | 9 | (16.9) |
| Other | 8 | (15.1) |
| Female, n (%) | 46 | (86.7) |
| Age (years), Median (IQR) | 26.70 | (22.3-42.0) |
| Duration SLE (years), Median (IQR) | 3.00 | (0.1-9.1) |
| Time from LN flare (months)*, Median (IQR) | 1.0 | (0-2.0) |
| SLEDAI, total score, Median (IQR) | 16.0 | (9.0-22.0) |
| SLEDAI, renal, Median (IQR) | 8.0 | (4.0-12.0) |
| Anti-dsDNA Ab (IU/ml), Median (IQR) | 77.0 | (9.0-100.0) |
| C3, g/rL, Median (IQR) | 0.66 | (0.52-0.82) |
| C4, gr/L, Median (IQR) | 0.07 | (0.07-0.21) |
| Serum Albumin (gr/L), Median (IQR) | 29.0 | (22.0-34.0) |
| Serum Creatinine (umol/L), Median (IQR) | 131.0 | (59.5-151.0) |
| 24-hour Protein excretion (gr), Median (IQR) | 3.02 | (1.3-3.6) |
| Kidney biopsy Class, n (%) | 53 | (100) |
| I | 1 | (1.8) |
| II | 3 | (5.6) |
| III | 4 | (7.5) |
| IV | 18 | (33.9) |
| V | 10 | (18.8) |
| III + V | 4 | (7.5) |
| IV + V | 13 | (24.5) |
| VI | 0 | (0) |
| Activity Index, Median (IQR) | 10.0 | (7.5-13.0) |
| Chronicity Index, Median (IQR) | 3.0 | (2.0-5.0) |
| Prednisone, n (%) | 52 | (98.1) |
| Prednisone dose (mg), Median (IQR) | 40.0 | (30.0-50.0) |
| Antimalarial, n (%) | 49 | (92.4) |
| Immunossuppressive, n (%) | 53 | (100) |
| Azathioprine, n (%) | 9 | (16.9) |
| Mychophenolate, n (%) | 34 | (64.1) |
| Cyclophosphamide, n (%) | 7 | (13.2) |
| Calcineurin Inhibitors, n (%) | 3 | (5.6) |

^{†}Baseline clinical characteristics are at the time of the urine sample collection,
*Time from LN flare to urine sample collection (months).

TABLE 7
Sensitivity of our “rule in ALN” criteria
in a biopsy-proven ALN cohort.

| | Whole cohort | Proliferative | Non-Proliferative |
|---|---|---|---|
| | (N = 53) | LN (N = 35) | LN (N = 18) |
| | Sensitivity (95% CI) using sVCAM cut-off of 46000 | | |
| 2 Elevated | 81.1 (68.0-90.6) | 91.4 (76.9-98.2) | 61.1 (35.8-82.7) |
| biomarkers | | | |
| 3 Elevated | 66.0 (51.3-78.8) | 77.1 (59.9-89.6) | 55.6 (30.8-78.5) |
| biomarkers | | | |
| | Sensitivity (95% CI) using sVCAM cut-off of 103,700 | | |
| 2 Elevated | 79.3 (65.9-89.2) | 91.4 (76.9-98.2) | 55.6 (30.8-78.5) |
| biomarkers | | | |
| 3 Elevated | 56.6 (42.3-70.2) | 62.9 (44.9-78.5) | 55.6 (30.8-78.5) |
| biomarkers | | | |

### CITATIONS FOR REFERENCES REFERRED TO IN THE SPECIFICATION

1. Maningding E, Dall'Era M, Trupin L, Murphy L B, Yazdany J. Racial and Ethnic Differences in the Prevalence and Time to Onset of Manifestations of Systemic Lupus Erythematosus: The California Lupus Surveillance Project. Arthritis care & research. 2020; 72 (5): 622-9.
2. Parikh S V, Almaani S, Brodsky S, Rovin B H. Update on Lupus Nephritis: Core Curriculum 2020. Am J Kidney Dis. 2020; 76 (2): 265-81.
3. Hanly J G, O'Keeffe A G, Su L, Urowitz M B, Romero-Diaz J, Gordon C, et al. The frequency and outcome of lupus nephritis: results from an international inception cohort study. Rheumatology. 2016; 55 (2): 252-62.
4. Hanly J G, O'Keeffe A G, Su L, Urowitz M B, Romero-Diaz J, Gordon C, et al. The frequency and outcome of lupus nephritis: results from an international inception cohort study. Rheumatology. 2016; 55 (2): 252-62.
5. Yurkovich M, Vostretsova K, Chen W J, Avina-Zubieta J A. Overall and Cause-Specific Mortality in Patients With Systemic Lupus Erythematosus: A Meta-Analysis of Observational Studies. Arthritis care & research. 2014; 66 (4): 608-16.
6. Mok C C, Kwok R C L, Yip P S F. Effect of Renal Disease on the Standardized Mortality Ratio and Life Expectancy of Patients With Systemic Lupus Erythematosus. Arthritis and rheumatism. 2013; 65 (8): 2154-60.
7. Yap D Y H, Tang C S O, Ma M K M, Lam M F, Chan T M. Survival analysis and causes of mortality in patients with lupus nephritis. Nephrology Dialysis Transplantation. 2012; 27 (8): 3248-54.
8. Lerang K, Gilboe I M, Thelle D S, Gran J T. Mortality and years of potential life loss in systemic lupus erythematosus: a population-based cohort study. Lupus. 2014; 23 (14): 1546-52.
9. Danila M I, Pons-Estel G J, Zhang J, Vila L M, Reveille J D, Alarcon G S. Renal damage is the most important predictor of mortality within the damage index: data from LUMINA LXIV, a multiethnic US cohort. Rheumatology. 2009; 48 (5): 542-5.
10. Chen Y E, Korbet S M, Katz R S, Schwartz M M, Lewis E J, Collaborative Study G. Value of a complete or partial remission in severe lupus nephritis. Clinical Journal of the American Society of Nephrology. 2008; 3 (1): 46-53.
11. Li T, Carls G S, Panopalis P, Wang S, Gibson T B, Goetzel R Z. Long-Term Medical Costs and Resource Utilization in Systemic Lupus Erythematosus and Lupus Nephritis: A Five-Year Analysis of a Large Medicaid Population. Arthritis & Rheumatism-Arthritis Care & Research. 2009; 61 (6): 755-63.
12. Tektonidou M G, Dasgupta A, Ward M M. Risk of End-Stage Renal Disease in Patients With Lupus Nephritis, 1971-2015 A Systematic Review and Bayesian Meta-Analysis. Arthritis & Rheumatology. 2016; 68 (6): 1432-41.
13. Weening J J, D'Agati V D, Schwartz M M, Seshan S V, Alpers C E, Appel G B, et al. The classification of glomerulonephritis in systemic lupus erythematosus revisited. Kidney international. 2004; 65 (2): 521-30.
14. Touma Z, Urowitz M B, Ibanez D, Gladman D D. Time to recovery from proteinuria in patients with lupus nephritis receiving standard treatment. The Journal of rheumatology. 2014; 41 (4): 688-97.
15. Malvar A, Pirruccio P, Alberton V, Lococo B, Recalde C, Fazini B, et al. Histologic versus clinical remission in proliferative lupus nephritis. Nephrol Dial Transplant. 2017; 32 (8): 1338-44.
16. Rovin B H, Song H, Hebert L A, Nadasdy T, Nadasdy G, Birmingham D J, et al. Plasma, urine, and renal expression of adiponectin in human systemic lupus erythematosus. Kidney international. 2005; 68 (4): 1825-33.
17. Landolt-Marticorena C, Prokopec S D, Morrison S, Noamani B, Bonilla D, Reich H, et al. A discrete cluster of urinary biomarkers discriminates between active systemic lupus erythematosus patients with and without glomerulonephritis. Arthritis research & therapy. 2016; 18 (1): 218.
18. Gamez-Nava J I, Diaz-Rizo V, Perez-Guerrero E E, Munoz-Valle J F, Saldana-Cruz A M, Fajardo-Robledo N S, et al. Assessment of serum macrophage migration inhibitory factor (MIF), adiponectin, and other adipokines as potential markers of proteinuria and renal dysfunction in lupus nephritis: a cross-sectional study. Biomarker research. 2020; 8:55.
19. Lee Y H, Song G G. Urinary MCP-1 as a biomarker for lupus nephritis: a meta-analysis. Zeitschrift fur Rheumatologie. 2017; 76 (4): 357-63.
20. Gomez-Puerta J A, Ortiz-Reyes B, Urrego T, Vanegas-Garcia A L, Munoz C H, Gonzalez L A, et al. Urinary neutrophil gelatinase-associated lipocalin and monocyte chemoattractant protein 1 as biomarkers for lupus nephritis in Colombian SLE patients. Lupus. 2018; 27 (4): 637-46.
21. Gupta R, Yadav A, Aggarwal A. Longitudinal assessment of monocyte chemoattractant protein-1 in lupus nephritis as a biomarker of disease activity. Clinical rheumatology. 2016; 35 (11): 2707-14.
22. Stanley S, Vanarsa K, Soliman S, Habazi D, Pedroza C, Gidley G, et al. Comprehensive aptamer-based screening identifies a spectrum of urinary biomarkers of lupus nephritis across ethnicities. Nat Commun. 2020; 11 (1): 2197.
23. Diaz-Rizo V, Bonilla-Lara D, Gonzalez-Lopez L, Sanchez-Mosco D, Fajardo-Robledo N S, Perez-Guerrero E E, et al. Serum levels of adiponectin and leptin as biomarkers of proteinuria in lupus nephritis. PLOS One. 2017; 12 (9): e0184056.
24. Hutcheson J, Ye Y, Han J, Arriens C, Saxena R, Li Q Z, et al. Resistin as a potential marker of renal disease in lupus nephritis. Clin Exp Immunol. 2015; 179 (3): 435-43.
25. Wofsy D, Hillson J L, Diamond B. Comparison of alternative primary outcome measures for use in lupus nephritis clinical trials. Arthritis and rheumatism. 2013; 65 (6): 1586-91.
26. Aitekenov S, Gaipov A, Bukassov R. Review: Detection and quantification of proteins in human urine. Talanta 2021; 223 121718

1. A urinary biomarker panel for detecting active Lupus Nephritis (ALN) comprising a plurality of biomarker detection agents, wherein each biomarker detection agent is specific for a corresponding target biomarker, the target biomarkers consisting of Adiponectin, MCP-1, sVCAM-1, and PF4, and none, one or both of vWF and IL-15, and optionally one or more control biomarkers.

2. The biomarker panel of claim 1, wherein each biomarker detection agent is an antibody or binding fragment thereof and/or further comprising a solid support.

3. (canceled)

4. The biomarker panel of claim 2, wherein the solid support is a bead, a plate, or a chip, optionally wherein the panel is configured as a multiplex assay or an ELISA assay.

5. The biomarker panel of claim 4, wherein the bead comprises a unique code, optionally a colour code, and each unique code is associated with a different biomarker detection agent.

6. A kit comprising the biomarker panel of claim 1, and one or more of:
i) a sample dilution buffer;
ii) a wash buffer;
iii) a filter;
iv) a positive control; and/or
v) instructions for performing a method described herein.

7. A device comprising:
i) a sample inlet configured for receiving one or more test urine samples; and
ii) a urinary biomarker panel, optionally the biomarker panel of any one of claims 1 to 5, configured to contact the test samples comprising a plurality of biomarker detection agents, wherein each biomarker detection agent is specific for a corresponding target biomarker selected from the group consisting of Adiponectin, MCP-1, sVCAM-1, and PF4, and none, one or both of vWF and IL-15, and optionally one or more control biomarkers.

8. (canceled)

9. A method of:
A) assessing treatment response and/or predicting a treatment outcome in a subject that has active Lupus Nephritis (ALN) comprising:
i) acquiring one or more test urine samples from the subject, wherein the test samples are acquired within about 6 to about 15 months following onset of administration of an ALN treatment or LN flare,
ii) measuring levels of a plurality of target biomarkers in the test samples, comprising contacting the test samples with a urinary biomarker panel comprising a plurality of biomarker detection agents, optionally the urinary biomarker panel of claim 1, wherein each biomarker detection agent is specific for a corresponding target biomarker and the plurality of target biomarkers comprise Adiponectin, MCP-1, sVCAM-1, and PF4, and none, one or both of vWF and IL-15;
iii) comparing the measured levels of each of the test sample target biomarkers to a control, optionally a base line level or cut-off level; and
iv) assessing response or predicting the treatment outcome,
wherein the subject is having or has an increased probability of having a complete response, to the treatment if the measured levels for at least two, at least three or each target biomarker are decreased compared to the control and/or less than two target biomarkers are decreased compared to the control optionally reach a cut-off level; or
B) detecting active Lupus Nephritis (ALN) in a subject comprising:
i) acquiring one or more test urine samples from the subject:
ii) measuring levels of a plurality of target biomarkers in the test samples, comprising contacting the test samples with a urinary biomarker panel comprising a plurality of biomarker detection agents, optionally the urinary biomarker panel of claim 1, wherein each biomarker detection agent is specific for a corresponding target biomarker and the plurality of target biomarkers comprise Adiponectin, MCP-1, sVCAM-1, and PF4, and none, one or both of vWF and IL-15;
iii) comparing the measured levels of each of the test sample target biomarkers to a cut-off level for each target biomarker; and
iv) detecting whether the subject has ALN or does not have ALN,
wherein if at least two of the target biomarkers are Adiponectin, MCP-1, sVCAM-1 and/or PF4 and the measured levels of the two biomarkers do not reach the cut-off levels, and the cut off level for sVCAM-1 is a threshold level, the subject is identified as likely not having ALN; and
if at least two of the target biomarkers are Adiponectin, MCP-1, sVCAM-1 and/or PF4 and the measured levels of the said at least two biomarkers reach the cut-off levels, wherein if the target biomarkers that reach the cut-off are two biomarkers and the two biomarkers includes sVCAM-1 and not MCP-1, the cut-off level of sVCAM-1 is an elevated cut off level the subject is identified as likely having ALN.

10. The method of claim 9, method A) wherein,
if at least two of the target biomarkers are Adiponectin, MCP-1, sVCAM-1 and/or PF4 and the measured levels of the two biomarkers do not reach the cut-off levels, and the cut off level for sVCAM-1 is a threshold level, the subject is identified as having or likely to have a complete response to the treatment; and
if at least two of the target biomarkers are Adiponectin, MCP-1, sVCAM-1 and/or PF4 and the measured levels of the said at least two biomarkers reach the cut-off levels, wherein if the target biomarkers that reach the cut-off are two biomarkers and the two biomarkers includes sVCAM-1 and not MCP-1, the cut-off level of sVCAM-1 is an elevated cut off level, the subject is identified as less likely to have a complete response to the treatment.

11. The method of claim 9, wherein the control is a cut-off level determined from levels from one or more subjects having ALN and known treatment outcomes and/or healthy control subjects and/or wherein the treatment outcome is at about 24 months.

12. (canceled)

13. The method of claim 9, wherein the cut-off level for each target biomarker is:
a) about 18 ng/ml for Adiponectin or about or at least 3 fold over a SLE NLN control population level;
b) about 1.3 ng/ml for MCP-1 or about or at least 5 fold over a SLE NLN control population level;
c) about 46 ng/ml for sVCAM-1 or about or at least 2 fold over a SLE NLN control population level; and/or
d) about 0.13 ng/ml for PF4 or about or at least 2.3 fold over a SLE NLN control population level.

14. The method of claim 9, wherein at least three of the target biomarkers are Adiponectin, MCP-1, sVCAM-1 and/or PF4 and measured levels for each of the said three target biomarkers reach the cut-off levels and indicate the subject likely has ALN or is unlikely to have a complete response to treatment.

15. The method of claim 9, wherein the at least two of the target biomarkers are:
A Adiponectin and MCP-1;
B Adiponectin and PF4;
C MCP-1 and PF4; or
D MCP-1 and sVCAM-1; or
wherein the at least two of the target biomarkers are:
e) sVCAM-1 and Adiponectin; or
f) sVCAM-1 and PF4; and
the cut off level for sVCAM-1 is the elevated cut-off level.

16. (canceled)

17. The method of claim 9, wherein the cut-off level for sVCAM-1 is about 104 ng/ml or about or at least 4.4 fold over a SLE NLN control population level and/or The method of any one of claims 9 to 24, wherein the ALN is proliferative Lupus nephritis (PLN).

18. The method of claim 9 method B, wherein the test samples is taken from a subject that has had a Lupus Nephritis (LN) flare within 12 or within 15 months from when the test samples were acquired and/or when the subject is predicted to have ALN, the method further comprises administering a treatment for ALN to the subject.

19. A method of treating active Lupus Nephritis (ALN) comprising:
in a subject receiving an ALN treatment, adjusting the ALN treatment in a subject having increased or comparable measured levels of at least two of a plurality of target biomarkers relative to a baseline level and/or control in one or more test urine samples, optionally predicted according to the method of claim 9 method A) as less likely to have a complete response, or continuing administration of the ALN treatment in a subject that has decreased measured levels of at least two of a plurality of target biomarkers relative to a baseline level and/or control in one or more test urine samples optionally predicted according to the method of any one of claims 9-11 as likely to have a complete response, wherein the target biomarkers comprise Adiponectin, MCP-1, sVCAM-1, and PF4, and none, one or both of vWF and IL-15 and wherein the measured levels are from samples taken within 6 to 15 months from onset of the treatment, optionally wherein adjusting the ALN treatment comprises discontinuing administration of the ALN treatment and initiating administration of an alternate ALN treatment, adjusting a dose of a component of the ALN treatment or adding a therapeutic; or
in a subject not receiving treatment for ALN, administering an ALN treatment, optionally comprising azathioprine, cyclophosphamide or mycophenolate, to a subject having measured levels of at least two target biomarkers of a plurality of biomarkers reach a cut-off level for each of the said at least two target biomarkers, wherein the target biomarkers comprise Adiponectin, MCP-1, sVCAM-1 and/or PF4, and none, one or both of vWF and IL-15.

20. The method of claim 19, wherein the test samples are taken at about 6, or about 9 or about 12 or about 15 months from onset of the treatment.

21. A method of monitoring and/or treating a subject with or suspected of having active Lupus Nephritis (ALN) comprising:
i) acquiring one or more test urine samples from the subject;
ii) measuring levels of a plurality of target biomarkers in the test samples, comprising contacting the test samples with a urinary biomarker panel comprising a plurality of biomarker detection agents, optionally the urinary biomarker panel of claim 1, wherein each biomarker detection agent is specific for a corresponding target biomarker and the plurality of target biomarkers comprise Adiponectin, MCP-1, sVCAM-1, and PF4, and none, one or both of vWF and IL-15, and optionally one or more control biomarkers;
iii) comparing the measured levels of each of the test sample target biomarkers to levels of the target biomarkers in one or more control samples, optionally wherein the control samples are baseline samples acquired from the subject or are acquired from one or more healthy control subjects; and
iv) a) administering an ALN treatment based on the measured levels of the target biomarkers in the test samples, wherein at least two of the target biomarkers are Adiponectin, MCP-1, sVCAM-1 and/or PF4 and the measured levels for the said target biomarkers reach a cut-off level for each target biomarker, optionally wherein the treatment comprises administering azathioprine, cyclophosphamide or mycophenolate; or
b) adjusting the ALN treatment, optionally discontinuing administration of the ALN treatment and administering an alternate ALN treatment based on the measured levels of the target biomarkers in the test samples, wherein at least two of the target biomarkers are Adiponectin, MCP-1, sVCAM-1 and/or PF4 and the measured levels for the said target biomarkers reach a cut-off level for each target biomarker; and/or
c) directing a follow up testing repeating steps i) to iii) to monitor the subject.

22. The method of claim 19, wherein the adjusted treatment or the alternate treatment comprises tacrolimus, voclosporin and/or belimumab.

23. The method of any one of claims 19 to 22, wherein the cut-off level for each target biomarker is:
a) about 18 ng/ml for Adiponectin or about or at least 3 fold over a SLE NLN control population level;
b) about 1.3 ng/ml for MCP-1 or about or at least 5 fold over a SLE NLN control population level;
c) about 46 ng/ml for sVCAM-1 or about or at least 2 fold over a SLE NLN control population level; and/or
d) about 0.13 ng/mL for PF4 or about or at least 2.3 fold over a SLE NLN control population level;
wherein at least three of the target biomarkers are Adiponectin, MCP-1, sVCAM-1 and/or PF4 and measured levels for each of the said three target biomarkers reach the cut-off levels and indicate adjusting the ALN treatment and/or administering the ALN treatment;
or wherein the at least two target biomarkers are:
a Adiponectin and MCP-1;
b Adiponectin and PF4;
c MCP-1 and PF4; or
d MCP-1 and sVCAM-1; or
the target biomarkers are:
i) sVCAM-1 and Adiponectin; or
k) sVCAM-1 and PF4; and
the cut off level for sVCAM-1 is the elevated cut-off level, optionally wherein the cut-off level for sVCAM-1 is about 104 ng/ml or about or at least 4.4 fold over a SLE NLN control population level.

24. (canceled)

25. (canceled)

26. The method of claim 9, wherein the cut-off level of each target biomarker is selected to provide a desired sensitivity, specificity, positive predictive value (PPV), negative predictive value (NPV), positive likelihood ratio (+LR) and/or negative likelihood ratio (−LR), optionally wherein the selected specificity, and/or NPV for each target biomarker is above about 90% or about 95%, and/or the PPV is above about 95% or about 100%.

27. (canceled)
